(19) United States
(12) Patent Application Publication
(10) Pub. No.: US 2025/0231731 A1
(43) Pub. Date: Jul. 17, 2025

(54) METHOD FOR INTERACTION BETWEEN WEARABLE DEVICE AND ELECTRONIC DEVICE, AND WEARABLE DEVICE AND ELECTRONIC DEVICE PERFORMING SAME

(71) Applicant: Samsung Electronics Co., Ltd., Suwon-si (KR)

(72) Inventors: Sukwon CHOI, Suwon-si (KR); Kyunghun JANG, Suwon-si (KR); Kiwan LEE, Suwon-si (KR); Jisu KIM, Suwon-si (KR); Myeongsang YU, Suwon-si (KR); Jiyeon LEE, Suwon-si (KR); Heeyoung CHO, Suwon-si (KR); Younghoon CHO, Suwon-si (KR)

(21) Appl. No.: 19/172,107

(22) Filed: Apr. 7, 2025

Related U.S. Application Data
(63) Continuation of application No. PCT/KR2023/014961, filed on Sep. 27, 2023.

(30) Foreign Application Priority Data
Oct. 7, 2022 (KR) 10-2022-0129015
Nov. 16, 2022 (KR) 10-2022-0153460
Dec. 12, 2022 (KR) 10-2022-0173160
Dec. 16, 2022 (KR) 10-2022-0177514

(51) Int. Cl. G06F 3/16; G06F 3/01

(52) U.S. Cl. CPC G06F 3/16; G06F 3/011; G06F 3/017

(57) ABSTRACT

Disclosed are a method for interaction between a wearable device and an electronic device, and the wearable device and electronic device performing same. The electronic device may include: a communication module for communicating with a wearable device; and a processor for controlling a user's exercise performed using the wearable device. The processor outputs first guide speech for guiding the proper wearing of the wearable device when a connection with the wearable device is detected, and outputs second guide speech for guiding an operation for sensor initialization of the wearable device in response to receiving, from the wearable device, first notification data indicating that the user has worn the wearable device properly. The processor outputs third guide speech for sequentially inquiring whether to select each of recommended exercise programs in response to receiving second notification data indicating that the sensor initialization has been completed from the wearable device, and executes an exercise mode according to a recommended target exercise program in response to receiving response data, corresponding to the selection of the recommended target exercise program among the recommended exercise programs, from the wearable device.

### CROSS-REFERENCE TO RELATED APPLICATIONS

This application is a continuation application of International Application No. PCT/KR2023/014961 designating the United States, filed on Sep. 27, 2023, in the Korean Intellectual Property Receiving Office and claiming priority to Korean Patent Application No. 10-2022-0129015, filed on Oct. 7, 2022, Korean Patent Application No. 10-2022-0153460, filed on Nov. 16, 2022, Korean Patent Application No. 10-2022-0173160, filed on Dec. 12, 2022, and Korean Patent Application No. 10-2022-0177514, filed on Dec. 16, 2022, the disclosures of which are all hereby incorporated by reference herein in their entireties.

### BACKGROUND

### Technical Field

Certain example embodiments may relate to a method of interaction between a wearable device, an electronic device, and/or a wearable device and/or an electronic device for performing the same.

### Background Art

A walking assistance device generally refers to a machine or a device that helps a patient unable to walk on their own because of diseases, accidents, or other causes with their walking exercises for rehabilitation treatment, and/or to a machine or device that helps a person exercise. In our current, rapidly aging society, a growing number of people experience inconvenience when walking or have difficulty with normal walking due to malfunctioning joints, and there is increasing interest in walking assistance devices. A walking assistance device may be worn on a body of a user to assist the user with walking by providing muscular strength and to induce the user to walk in a normal walking pattern.

### SUMMARY

An electronic device according to an example embodiment may include a communication module, comprising communication circuitry, configured to communicate with a wearable device, and at least one processor, comprising processing circuitry, individually and/or collectively configured to control an exercise of a user performed using the wearable device based on the communication with the wearable device. The at least one processor may output a first guide voice for guiding proper wearing of the wearable device when connection to the wearable device is detected. The at least one processor may output a second guide voice for guiding an operation for sensor initialization of the wearable device after first notification data indicating that the user is properly wearing the wearable device is received from the wearable device. The at least one processor may output a third guide voice for sequentially inquiring whether to select each one of a plurality of recommended exercise programs in response to reception of second notification data indicating that the sensor initialization is completed from the wearable device. The at least one processor may execute an exercise mode according to a target recommended exercise program in response to reception of response data corresponding to selection of the target recommended exercise program among the recommended exercise programs from the wearable device.

An example interaction method between an electronic device and a wearable device, performed by the electronic device according to an embodiment may include outputting a first guide voice for guiding proper wearing of the wearable device when connection to the wearable device is detected. The interaction method may further include receiving first notification data indicating that a user is properly wearing the wearable device from the wearable device, and outputting a second guide voice for guiding an operation for sensor initialization of the wearable device in response to the reception of the first notification data. The interaction method may further include receiving second notification data indicating that the sensor initialization is completed from the wearable device, and outputting a third guide voice for sequentially inquiring whether to select each one of a plurality of recommended exercise programs in response to the reception of the second notification data. The interaction method may further include executing an exercise mode according to a target recommended exercise program when response data corresponding to selection of the target recommended exercise program among the recommended exercise programs is received from the wearable device.

According to an example embodiment, a non-transitory computer-readable storage medium storing instructions that, when executed by a processor, may cause the processor to perform the interaction method.

### BRIEF DESCRIPTION OF DRAWINGS

FIG. 1 is a diagram illustrating an overview of a wearable device worn on a body of a user according to an example embodiment.

FIG. 2 is a diagram illustrating an exercise management system including a wearable device and an electronic device according to an example embodiment.

FIG. 3 is a rear schematic view illustrating a wearable device according to an example embodiment.

FIG. 4 is a left side view illustrating a wearable device according to an example embodiment.

FIGS. 5A and 5B are diagrams illustrating a configuration of a control system of a wearable device according to an example embodiment.

FIG. 6 is a diagram illustrating an interaction between a wearable device and an electronic device according to an example embodiment.

FIG. 7 is a diagram illustrating a configuration of an electronic device according to an example embodiment.

FIGS. 8A and 8B are flowcharts illustrating interaction methods between a wearable device and an electronic device in an exercise preparation and exercise starting operation according to an example embodiment.

FIG. 9 is a flowchart illustrating an interaction method between a wearable device and an electronic device in an exercise progress and exercise completion operation according to an example embodiment.

FIG. 10 is a diagram illustrating determination of a recommended exercise program to be provided to a user according to an example embodiment.

FIG. 11 is a diagram illustrating decision making in a multi-device environment according to an example embodiment.

FIGS. 12A and 12B are diagrams illustrating examples in which guide voices are provided in a multi-device environment according to an example embodiment(s).

FIGS. 13A, 13B, 13C, and 13D are diagrams illustrating a voice coaching/guidance function provided during an exercise process of a user according to an example embodiment(s).

FIGS. 14A, 14B, and 14C are diagrams illustrating a voice coaching/guidance function provided for each set constituting an exercise program according to an example embodiment(s).

FIGS. 15A, 15B, 15C, 15D, 15E, 15F, 15G, 15H, and 15I are diagrams illustrating a voice coaching/guidance function provided for each exercise operation and/or situation of a user according to an example embodiment(s).

### DETAILED DESCRIPTION

The following detailed structural or functional description is provided as an example only and various alterations and modifications may be made to the examples. Accordingly, the example embodiments are not construed as limited to the disclosure and should be understood to include all changes, equivalents, and replacements within the idea and the technical scope of the disclosure.

The singular forms “a”, “an”, and “the” are intended to include the plural forms as well, unless the context clearly indicates otherwise. It will be further understood that the terms “comprises/comprising” and/or “includes/including” when used herein, specify the presence of stated features, integers, steps, operations, elements, and/or components, but do not preclude the presence or addition of one or more other features, integers, steps, operations, elements, components and/or groups thereof.

Unless otherwise defined, all terms, including technical and scientific terms, used herein have the same meaning as commonly understood by one of ordinary skill in the art to which this disclosure pertains. Terms, such as those defined in commonly used dictionaries, should be construed to have meanings matching with contextual meanings in the relevant art, and are not to be construed to have an ideal or excessively formal meaning unless otherwise defined herein.

Hereinafter, embodiments will be described in detail with reference to the accompanying drawings. When describing the examples with reference to the accompanying drawings, like reference numerals refer to like elements and a repeated description related thereto will be omitted.

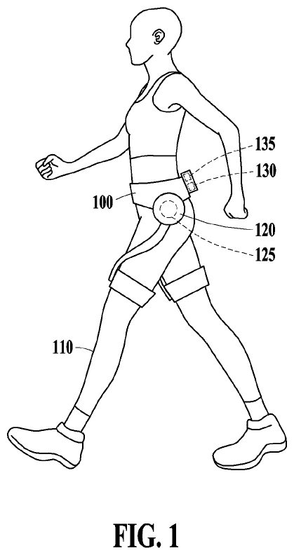

FIG. 1 is a diagram illustrating an overview of a wearable device worn on a body of a user according to an embodiment.

Referring to FIG. 1, in an embodiment, a wearable device 100 may be a device worn on a body of a user 110 to assist the user 110 in walking, exercising, and/or working. In an embodiment, the wearable device 100 may be used to measure a physical ability (e.g., a walking ability, an exercise ability, or an exercise posture) of the user 110. In embodiments, the term “wearable device” may be replaced with “wearable robot,” “walking assistance device,” or “exercise assistance device”. The user 110 may be a human or an animal, but is not limited thereto. The wearable device 100 may be worn on a body (e.g., a lower body (the legs, ankles, knees, etc.), an upper body (the torso, arms, wrists, etc.), or the waist) of the user 110 to provide an external force such as an assistance force and/or a resistance force to a body motion of the user 110. The resistance force may be a force applied in the same direction as the body motion direction of the user 110, the force assisting a body motion of the user 110. The resistance force may be a force applied in a direction opposite to the body motion direction of the user 110, the force hindering a body motion of the user 110. The term “resistance force” may also be referred to as “exercise load”.

In an embodiment, the wearable device 100 may operate in a walking assistance mode for assisting the user 110 in walking. In the walking assistance mode, the wearable device 100 may assist the user 110 in walking by applying an assistance force generated by a driving module 120 of the wearable device 100 to the body of the user 110. The wearable device 100 may enable the user 110 to walk independently or to walk for a long time by providing a force required for the user 110 to walk, thereby extending the walking ability of the user 110. The wearable device 100 may help in improving an abnormal walking habit or walking posture of a walker.

In an embodiment, the wearable device 100 may operate in an exercise assistance mode for enhancing the exercise effect of the user 110. In the exercise assistance mode, the wearable device 100 may hinder a body motion of the user 110 or provide resistance to a body motion of the user 110 by applying a resistance force generated by the driving module 120 to the body of the user 110. When the wearable device 100 is a hip-type wearable device that is worn on the waist (or pelvis) and legs (e.g., thighs) of the user 110, the wearable device 100 may provide an exercise load to a leg motion of the user 110 while being worn on the legs, thereby enhancing the exercise effect on the legs of the user 110. In an embodiment, the wearable device 100 may apply an assistance force to the body of the user 110 to assist the user 110 in exercising. For example, when a person with disabilities or an elderly person wants to exercise wearing the wearable device 100, the wearable device 100 may provide an assistance force for assisting a body motion during the exercise process. In an embodiment, the wearable device 100 may provide an assistance force and a resistance force in combination for each exercise section or time section, in such a manner of providing an assistance force in some exercise sections and a resistance force in other exercise sections.

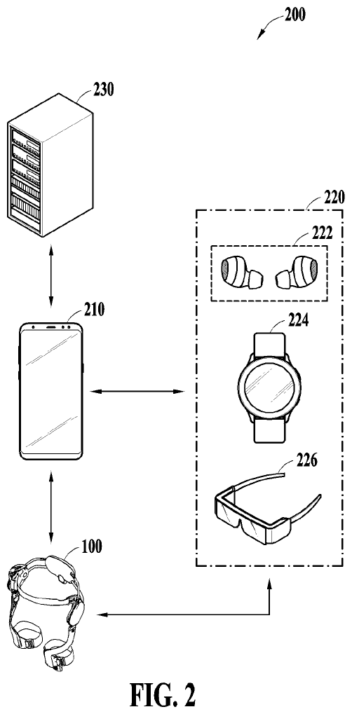

In an embodiment, the wearable device 100 may operate in a physical ability measurement mode (or an exercise ability measurement mode) to measure (including measurement of an exercise ability) a physical ability of the user 110. The wearable device 100 may measure motion information of the user by using one or more sensors (e.g., an angle sensor 125 and an inertial measurement unit (IMU) (or an inertial sensor) 135) included in the wearable device 100 while the user 110 performs walking and exercise, and the wearable device 100 or an electronic device (e.g., an electronic device 210 of FIG. 2) interworking with the wearable device 100 may evaluate a physical ability or an exercise ability of the user based on the measured motion information. For example, a gait index or an exercise ability indicator (e.g., the muscular strength, endurance, balance, or exercise posture) of the user 110 may be estimated through the motion information of the user 110 measured by the wearable device 100. The physical ability measurement mode may include an exercise posture evaluation mode to evaluate exercise posture (or exercise motion) of the user while the user performs exercise.

In various embodiments of the present disclosure, for convenience of description, the wearable device 100 is described as an example of a hip-type wearable device, as illustrated in FIG. 1, but the embodiments are not limited thereto. As described above, the wearable device 100 may be worn on another body part (e.g., the upper arms, lower arms, hands, calves, and feet) other than the waist and legs (particularly, the thighs), and the shape and configuration of the wearable device 100 may vary depending on the body part on which the wearable device 100 is worn.

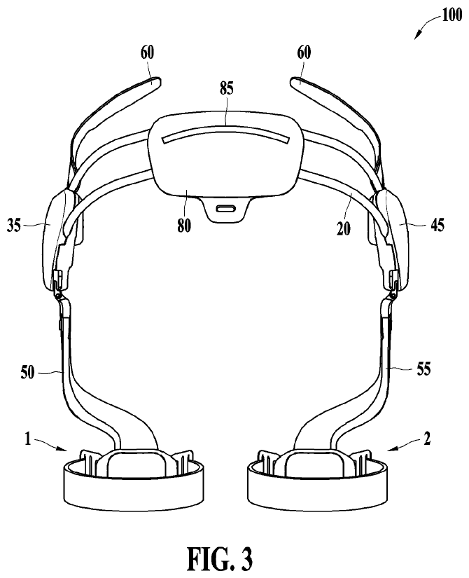

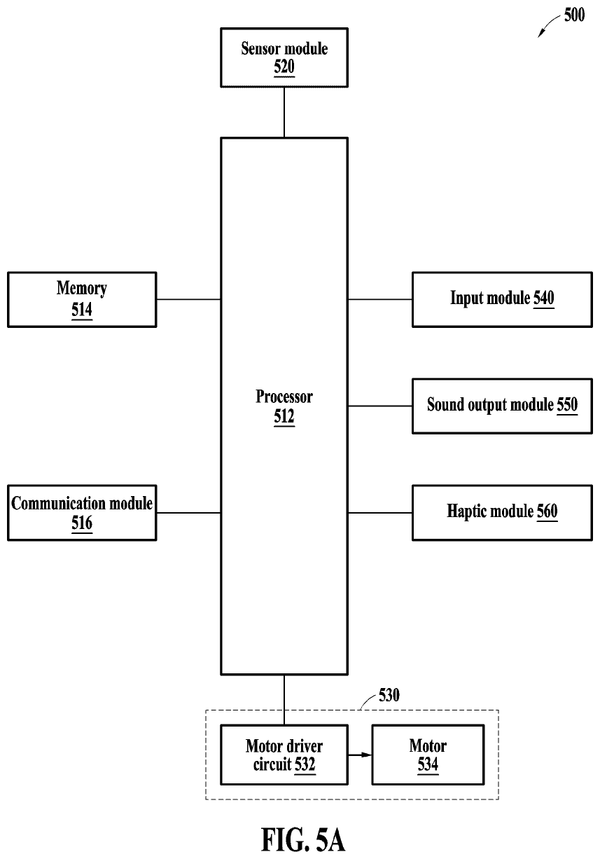

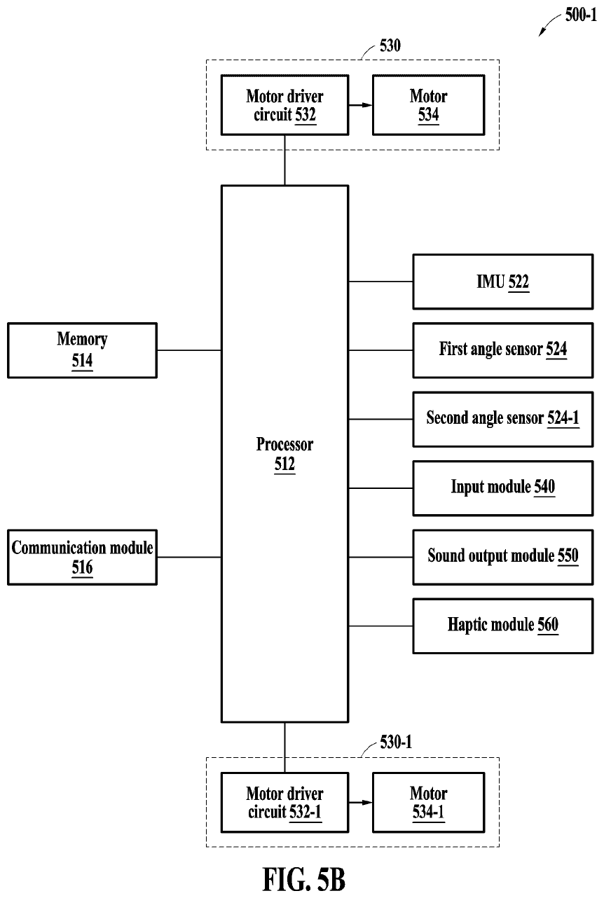

According to an embodiment, the wearable device 100 may include a support frame (e.g., a waist support frame 20 of FIG. 3) for supporting the body of the user 110 when the wearable device 100 is worn on the body of the user 110, the driving module 120 (e.g., driving modules 35 and 45 of FIG. 3) for generating torque applied to the legs of the user 110, a leg driving frame (e.g., leg driving frames 50 and 55 of FIG. 3) for transferring the torque generated by the driving module 120 to the legs of the user 110, a sensor module (e.g., a sensor module 520 of FIG. 5A) including one or more sensors for obtaining sensor data including motion information on a body motion (e.g., a leg motion or upper body motion) of the user 110, and a processor 130 for controlling an operation of the wearable device 100.

The sensor module may include the angle sensor 125 and the IMU 135. The angle sensor 125 may measure a rotational angle of a leg driving frame of the wearable device 100 corresponding to a hip joint angle value of the user 110. The rotational angle of the leg driving frame measured by the angle sensor 125 may be estimated as the hip joint angle value (or leg angle value) of the user 110. The angle sensor 125 may include, for example, an encoder and/or a Hall sensor. In an embodiment, the angle sensor 125 may be disposed in a vicinity where a motor included in the driving module 120 is connected, directly or indirectly, to the leg driving frame. The IMU 135 may include an acceleration sensor and/or an angular velocity sensor, and may measure a change in acceleration and/or angular velocity according to a motion of the user 110. The IMU 135 may measure a motion value of, for example, a waist support frame or a base body (e.g., a base body 80 of FIG. 3) of the wearable device 100. The motion value of the waist support frame or the base body measured by the IMU 135 may be estimated as the upper body motion value of the user 110. Herein, the term “IMU” may be replaced with “inertial sensor”.

In an embodiment, the processor 130 and the IMU 135 may be arranged within the base body (e.g., the base body 80 of FIG. 3) of the wearable device 100. The base body may be positioned on the lumbar region (an area of the lower back) of the user 110 while the user 110 is wearing the wearable device 100. The base body may be formed or attached to the outer side of the waist support frame of the wearable device 100. The base body may be mounted on, directly or indirectly, the lumbar region of the user 110 to provide a cushioning feeling to the lower back of the user 110 and may support the lower back of the user 110 together with the waist support frame.

Each “processor” herein includes processing circuitry, and/or may include multiple processors. For example, as used herein, including the claims, the term “processor” may include various processing circuitry, including at least one processor, wherein one or more of at least one processor, individually and/or collectively in a distributed manner, may be configured to perform various functions described herein. As used herein, when “a processor”, “at least one processor”, and “one or more processors” are described as being configured to perform numerous functions, these terms cover situations, for example and without limitation, in which one processor performs some of recited functions and another processor(s) performs other of recited functions, and also situations in which a single processor may perform all recited functions. Additionally, the at least one processor may include a combination of processors performing various of the recited/disclosed functions, e.g., in a distributed manner. At least one processor may execute program instructions to achieve or perform various functions.

FIG. 2 is a diagram illustrating an exercise management system including a wearable device and an electronic device according to an embodiment.

Referring to FIG. 2, an exercise management system 200 may include the wearable device 100, an electronic device 210, another wearable device 220, and a server 230. In an embodiment, at least one (e.g., the other wearable device 220 or the server 230) of these devices may be omitted from the exercise management system 200, or one or more other devices (e.g., an exclusive controller device of the wearable device 100) may be added to the exercise management system 200.

In an embodiment, the wearable device 100 may be worn on a body of a user to assist a motion of the user in a walking assistance mode. For example, the wearable device 100 may be worn on legs of the user to help the user in walking by generating an assistance force for assisting a leg motion of the user.

In an embodiment, the wearable device 100 may generate a resistance force for hindering a body motion of the user or an assistance force for assisting a body motion of the user and apply the generated resistance force or assistance force to the body of the user to enhance the exercise effect of the user in an exercise assistance mode. In the exercise assistance mode, the user may select, through the electronic device 210, an exercise program (e.g., squat, split lunge, dumbbell squat, lunge and knee up, stretching, or the like) to perform using the wearable device 100 and/or an exercise intensity to be applied to the wearable device 100. The wearable device 100 may control a driving module of the wearable device 100 according to the exercise program selected by the user and obtain sensor data including motion information of the user through a sensor module. The wearable device 100 may adjust the strength of the resistance force or assistance force applied to the user according to the exercise intensity selected by the user. For example, the wearable device 100 may control the driving module to generate a resistance force corresponding to the exercise intensity selected by the user.

In an embodiment, the wearable device 100 may be used to measure a physical ability of the user by interworking with the electronic device 210. This measurement of physical ability may include the measurement of an exercise ability. The wearable device 100 may operate in a physical ability measurement mode, which is a mode for measuring the physical ability of the user, under a control of the electronic device 210, and may transmit sensor data obtained by a motion of the user in the physical ability measurement mode to the electronic device 210. The wearable device 100 may transmit the motion data of the user to the electronic device 210 in real time. The electronic device 210 may evaluate the physical ability of the user by analyzing the sensor data received from the wearable device 100.

The electronic device 210 may communicate with the wearable device 100 and may remotely control the wearable device 100 or provide the user with state information about a state (e.g., a booting state, a charging state, a sensing state, or an error state) of the wearable device 100. The electronic device 210 may receive the sensor data obtained by the sensor module in the wearable device 100 from the wearable device 100 and estimate the physical ability of the user or an exercise result based on the received sensor data. In an embodiment, the electronic device 210 may provide the user with the physical ability of the user or the exercise result through a graphical user interface (GUI). “Based on” as used herein covers based at least on.

In an embodiment, the user may execute a program (e.g., an application) in the electronic device 210 to control the wearable device 100, and adjust an operation or a set value of the wearable device 100 (e.g., torque intensity output from a driving module (e.g., the driving modules 35 and 45 of FIG. 3), a volume of audio output from a sound output module (e.g., a sound output module 550 of FIGS. 5A and 5B), or a brightness of a lighting unit (e.g., a lighting unit 85 of FIG. 3) through the corresponding program. The program executed by the electronic device 210 may provide a GUI for interaction with the user. The electronic device 210 may be a device in various forms. For example, the electronic device 210 may include, but is not limited to, a portable communication device (e.g., a smartphone), a computer device, an access point, a portable multimedia device, or a home appliance device (e.g., a television, an audio device, a projector device).

According to an embodiment, the electronic device 210 may be connected to the server 230 using short-range wireless communication or cellular communication. The server 230 may receive user profile information of the user who uses the wearable device 100 from the electronic device 210 and store and manage the received user profile information. The user profile information may include, for example, information about at least one of the name, age, gender, height, weight, exercise goal, physical fitness level, medical history, or body mass index (BMI). The server 230 may receive exercise history information about an exercise performed by the user from the electronic device 210 and store and manage the received exercise history information. The server 230 may provide the electronic device 210 with various exercise programs or physical ability measurement programs that may be provided to the user. For example, the server 230 may be a cloud server. In an embodiment, the motion data measured by the wearable device 100 may be transmitted to the server 230 via the electronic device 210, and the server 230 may analyze the physical ability or exercise results of the user based on the motion data of the user. Result data derived from the analysis of the server 230 may be transmitted to the electronic device 210.

According to an embodiment, the wearable device 100 and/or the electronic device 210 may be connected to the other wearable device 220. The other wearable device 220 may include, for example, wireless earphones 222, a smartwatch 224, or smart glasses 226, but is not limited thereto. The wireless earphones 222 may be linked with the electronic device 210 to provide auditory feedback to the user. The wireless earphones 222 may output, for example, a guide voice for guiding the proper wearing of the wearable device 100 and for guiding actions for sensor initialization and/or a guide voice for real-time exercise coaching to the user. In an embodiment, the wireless earphones 222 may also function as a microphone for voice recognition. The user may control the electronic device 210 and the wearable device 100 through voice recognition. In an embodiment, the smartwatch 224 may measure a biosignal including heart rate information of the user and transmit the measured biosignal to the electronic device 210 and/or the wearable device 100. The electronic device 210 may estimate the heart rate information (e.g., a current heart rate, a maximum heart rate, and an average heart rate) of the user based on the biosignal received from the smartwatch 224 and provide the estimated heart rate information to the user. In an embodiment, the smartwatch 224 may output a guide screen for guiding exercise coaching and/or an exercise performance method to the user while the user is exercising.

In an embodiment, the exercise result information, physical ability information, and/or exercise posture evaluation information of the user determined by the electronic device 210 may be transmitted to the other wearable device 220 and provided to the user through the other wearable device 220. The state information of the wearable device 100 may also be transmitted to the other wearable device 220 and provided to the user through the other wearable device 220. For example, the exercise result information may be displayed on a screen of the smartwatch 224, or a guide voice for guiding the exercise result information may be output through the wireless earphones 222. In an embodiment, the wearable device 100, the electronic device 210, and the other wearable device 220 may be connected to each other through wireless communication (e.g., Bluetooth communication or wireless fidelity (Wi-Fi) communication).

In an embodiment, the wearable device 100 may provide (or output) feedback (e.g., visual feedback, auditory feedback, or haptic feedback) corresponding to the state of the wearable device 100 according to the control signal received from the electronic device 210. For example, the wearable device 100 may provide visual feedback through the lighting unit (e.g., the lighting unit 85 of FIG. 3) and provide auditory feedback through the sound output module (e.g., the sound output module 550 of FIGS. 5A and 5B). The wearable device 100 may provide haptic feedback in a form of vibration to the body of the user through a haptic module (e.g., a haptic module 560 of FIGS. 5A and 5B). The electronic device 210 may also provide (or output) feedback (e.g., visual feedback, auditory feedback, or haptic feedback) corresponding to the state of the wearable device 100. In an embodiment, the electronic device 210 may perform a voice-based exercise coaching function in real time while the user is exercising and may provide the user with relevant information through various feedback methods.

In an embodiment, the electronic device 210 may present a personalized exercise goal to the user in the exercise assistance mode or the physical ability measurement mode. The personalized exercise goal may include respective target amounts of exercise for exercise types (e.g., strength exercise, balance exercise, and aerobic exercise) desired by the user, determined by the electronic device 210 and/or the server 230. When the server 230 determines a target amount of exercise, the server 230 may transmit information about the determined target amount of exercise to the electronic device 210. The electronic device 210 may personalize and present the target amounts of exercise for the exercise types, such as strength exercise, aerobic exercise, and balance exercise, according to a desired exercise program (e.g., squat, split lunge, or a lunge and knee up) and/or physical characteristics (e.g., the age, height, weight, and BMI) of the user. The electronic device 210 may display a GUI screen indicating the target amount of exercise for each exercise type on a display or guide the user to the target amount of exercise through a guide voice.

In an embodiment, the electronic device 210 and/or the server 230 may include a database in which information about a plurality of exercise programs to be provided to the user through the wearable device 100 is stored. To achieve an exercise goal of the user, the electronic device 210 and/or the server 230 may recommend an exercise program suitable for the user. The exercise goal may include, for example, at least one of muscle strength improvement, physical strength improvement, cardiovascular endurance improvement, core stability improvement, flexibility improvement, or symmetry improvement. The electronic device 210 and/or the server 230 may store and manage the exercise program performed by the user, results of performing the exercise program, and the like. Exercise programs recommended to the user may be guided to the user through a guide voice or may be displayed through the GUI screen.

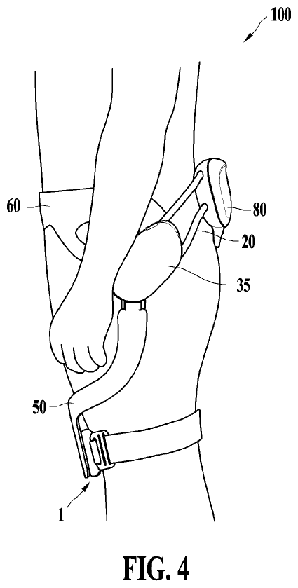

FIG. 3 is a rear schematic view illustrating a wearable device according to an embodiment. FIG. 4 is a left side view illustrating a wearable device according to an embodiment.

Referring to FIGS. 3 and 4, the wearable device 100 according to an embodiment may include the base body 80, the waist support frame 20, the driving modules 35 and 45, the leg driving frames 50 and 55, thigh fastening portions 1 and 2, and a waist fastening portion 60. The base body 80 may include the lighting unit 85. In an embodiment, at least one (e.g., the lighting unit 85) of the components described above may be omitted from the wearable device 100, or one or more other components may be added to the wearable device 100.

The base body 80 may be positioned on the lumbar region of a user while the user is wearing the wearable device 100. The base body 80 may be mounted on the lumbar region of the user to provide a cushioning feeling to the lower back of the user and may support the lower back of the user. The base body 80 may be hung on a hip region (an area of the hips) of the user to prevent or reduce chances of the wearable device 100 from being separated downward due to gravity while the user is wearing the wearable device 100. The base body 80 may distribute a portion of a weight of the wearable device 100 to the lower back of the user while the user is wearing the wearable device 100. The base body 80 may be connected, directly or indirectly, to the waist support frame 20. Waist support frame connecting elements (not shown) to be connected, directly or indirectly, to the waist support frame 20 may be provided at both end portions of the base body 80.

In an embodiment, the lighting unit 85 may be arranged on an outer surface of the base body 80. The lighting unit 85 may include a light source (e.g., a light emitting diode (LED)). The lighting unit 85 may emit light by control of a processor (not shown) (e.g., a processor 512 of FIGS. 5A and 5B). According to embodiments, the processor may control the lighting unit 85 to provide (or output) visual feedback corresponding to the state of the wearable device 100 through the lighting unit 85.

The waist support frame 20 may support the body (e.g., the waist) of the user when the wearable device 100 is worn on the body of the user. The waist support frame 20 may extend from both end portions of the base body 80. The lumbar region of the user may be accommodated inside the waist support frame 20. The waist support frame 20 may include at least one rigid body beam. Each beam may be in a curved shape having a preset curvature to enclose the lumbar region of the user. The waist fastening portion 60 may be connected, directly or indirectly, to an end portion of the waist support frame 20. The driving modules 35 and 45 may be connected, directly or indirectly, to the waist support frame 20.

In an embodiment, a processor, a memory, an IMU (e.g., the IMU 135 of FIG. 1 or an IMU 522 of FIG. 5B), a communication module (e.g., a communication module 516 of FIGS. 5A and 5B), a sound output module (e.g., the sound output module 550 of FIGS. 5A and 5B), and a battery (not shown) may be disposed in the base body 80. The base body 80 may protect the components disposed therein. The processor may generate a control signal for controlling an operation of the wearable device 100. The processor may control actuators of the driving modules 35 and 45. The processor and the memory may be included in a control circuit. The control circuit may further include a power supply circuit configured to supply power of the battery to each of the components of the wearable device 100.

In an embodiment, the wearable device 100 may include a sensor module (not shown) (e.g., the sensor module 520 of FIG. 5A) configured to obtain sensor data from at least one sensor. The sensor module may obtain sensor data including motion information of the user and/or motion information of a component of the wearable device 100. The sensor module may include, for example, an IMU (e.g., the IMU 135 of FIG. 1 or the IMU 522 of FIG. 5B) configured to measure an upper body motion value of the user or a motion value of the waist support frame 20 and an angle sensor (e.g., the angle sensor 125 of FIG. 1 or a first angle sensor 524 and a second angle sensor 524-1 of FIG. 5B) configured to measure a hip joint angle value of the user or a motion value of the leg driving frames 50 and 55, but is not limited thereto. For example, the sensor module may further include at least one of a position sensor, a temperature sensor, a biosignal sensor, or a proximity sensor.

The waist fastening portion 60 may be connected to the waist support frame 20 to fasten the waist support frame 20 to a waist of the user. The waist fastening portion 60 may include, for example, a pair of belts.

The driving modules 35 and 45 may generate an external force (or torque) to be applied to the body of the user based on the control signal generated by the processor.

For example, the driving modules 35 and 45 may generate an assistance force or resistance force to be applied to the legs of the user. In an embodiment, the driving modules 35 and 45 may include a first driving module 45 disposed in a position corresponding to a position of a right hip joint of the user, and a second driving module 35 disposed in a position corresponding to a position of a left hip joint of the user. The first driving module 45 may include a first actuator and a first joint member, and the second driving module 35 may include a second actuator and a second joint member. The first actuator may provide power to be transmitted to the first joint member, and the second actuator may provide power to be transmitted to the second joint member. The first actuator and the second actuator may each include a motor (e.g., motors 534 and 534-1 of FIG. 5B) configured to generate power (or torque) by receiving power from a battery. When the motor is supplied with electric power and driven, the motor may generate a force (an assistance force) for assisting a body motion of the user or a force (a resistance force) for hindering a body motion of the user. In an embodiment, the control module may adjust a strength and direction of the force generated by the motor by adjusting a voltage and/or a current supplied to the motor.

In an embodiment, the first joint member and the second joint member may receive power from the first actuator and the second actuator, respectively, and apply an external force to the body of the user based on the received power. The first joint member and the second joint member may be arranged at positions corresponding to joints of the user, respectively. One side of the first joint member may be connected to the first actuator, and the other side thereof may be connected to a first leg driving frame 55. The first joint member may be rotated by the power received from the first actuator. An encoder or a Hall sensor that may operate as an angle sensor to measure a rotation angle (corresponding to a joint angle of the user) of the first joint member or the first leg driving frame 55 may be disposed on one side of the first joint member. One side of the second joint member may be connected to the second actuator, and the other side thereof may be connected to a second leg driving frame 50. The second joint member may be rotated by the power received from the second actuator. An encoder or a hall sensor that may operate as an angle sensor to measure a rotation angle of the second joint member or the second leg driving frame 50 may be disposed on one side of the second joint member.

In an embodiment, the first actuator may be arranged in a lateral direction of the first joint member, and the second actuator may be arranged in a lateral direction of the second joint member. A rotation axis of the first actuator and a rotation axis of the first joint member may be spaced apart from each other, and a rotation axis of the second actuator and a rotation axis of the second joint member may also be spaced apart from each other. However, embodiments are not limited thereto, and an actuator and a joint member may share a rotation axis. In an embodiment, each actuator may be spaced apart from a corresponding joint member. In this case, each of the driving modules 35 and 45 may further include a power transmission module (not shown) configured to transmit power from the actuator to the joint member. The power transmission module may be a rotary body, such as a gear, or a longitudinal member, such as a wire, a cable, a string, a spring, a belt, or a chain. However, the scope of the embodiment is not limited by the positional relationship between an actuator and a joint member and the power transmission structure described above.

In an embodiment, the leg driving frames 50 and 55 may transmit torque generated by the driving modules 35 and 45 to the body (e.g., the thighs) of the user when the wearable device 100 is worn on the legs of the user. The transmitted torque may act as an external force applied to the motions of the legs of the user. As one end portions of the leg driving frames 50 and 55 are connected to the joint members to rotate and the other end portions of the leg driving frames 50 and 55 are connected to the thigh fastening portions 1 and 2, the leg driving frames 50 and 55 may transmit the torque generated by the driving modules 35 and 45 to the thighs of the user while supporting the thighs of the user. For example, the leg driving frames 50 and 55 may push or pull the thighs of the user. The leg driving frames 50 and 55 may extend in a longitudinal direction of the thighs of the user. The leg driving frames 50 and 55 may be bent to surround at least a portion of the circumference of the thighs of the user. The leg driving frames 50 and 55 may include the first leg driving frame 55 configured to transmit torque to the right leg of the user and the second leg driving frame 50 configured to transmit torque to the left leg of the user.

The thigh fastening portions 1 and 2 may be connected to the leg driving frames 50 and 55 and may fasten the wearable device 100 to the thighs of the user. For example, the thigh fastening portions 1 and 2 may include a first thigh fastening portion 2 configured to fasten the wearable device 100 to the right thigh of the user and a second thigh fastening portion 1 configured to fasten the wearable device 100 to the left thigh of the user.

In an embodiment, the first thigh fastening portion 2 may include a first cover, a first fastening frame, and a first strap, and the second thigh fastening portion 1 may include a second cover, a second fastening frame, and a second strap. The first cover and the second cover may apply torques generated by the driving modules 35 and 45 to the thighs of the user. The first cover and the second cover may be arranged on one sides of the thighs of the user to push or pull the thighs of the user. For example, the first cover and the second cover may be arranged on front surfaces of the thighs of the user. The first cover and the second cover may be arranged in circumferential directions of the thighs of the user. The first cover and the second cover may extend to both sides from the other end portions of the leg driving frames 50 and 55 and include curved surfaces corresponding to the thighs of the user. One ends of the first cover and the second cover may be connected to the fastening frames, and the other ends thereof may be connected to the straps.

The first fastening frame and the second fastening frame may be arranged, for example, to surround at least some portions of the circumferences of the thighs of the user, thereby preventing or reducing chances of the thighs of the user from being separated from the wearable device 100. The first fastening frame may have a fastening structure that connects the first cover and the first strap, and the second fastening frame may have a fastening structure that connects the second cover and the second strap.

The first strap may enclose the remaining portion of the circumference of the right thigh of the user that is not covered by the first cover and the first fastening frame, and the second strap may enclose the remaining portion of the circumference of the left thigh of the user that is not covered by the second cover and the second fastening frame. The first strap and the second strap may include, for example, an elastic material (e.g., a band).

FIGS. 5A and 5B are diagrams illustrating a configuration of a control system of a wearable device according to an embodiment.

Referring to FIG. 5A, the wearable device 100 may be controlled by a control system 500. The control system 500 may include the processor 512 (e.g., the processor 130), a memory 514, the communication module 516, the sensor module 520, a driving module 530, an input module 540, the sound output module 550, and the haptic module 560. In an embodiment, at least one (e.g., the sound output module 550 or the haptic module 560) of those components may be omitted from the control system 500, or one or more other components may be added to the control system 500.

The driving module 530 may include a motor 534 for generating torque (e.g., power) and a motor driver circuit 532 for controlling the motor 534. Although FIG. 5A illustrates the driving module 530 including one motor driver circuit 532 and one motor 534, the embodiment of FIG. 5A is merely an example. Referring to FIG. 5B, a control system 500-1 shown in FIG. 5B may include two or more (e.g., three or more) motor driver circuits 532 and 532-1 and motors 534 and 534-1. The driving module 530 including the motor driver circuit 532 and the motor 534 may correspond to the first driving module 45 of FIG. 3, and a driving module 530-1 including the motor driver circuit 532-1 and the motor 534-1 may correspond to the second driving module 35 of FIG. 3. The following descriptions of the motor driver circuit 532 and the motor 534 may also be respectively applicable to the motor driver circuit 532-1 and the motor 534-1 shown in FIG. 5B.

Referring back to FIG. 5A, the sensor module 520 may include a sensor circuit including at least one sensor. The sensor module 520 may include sensor data including motion information of components of the wearable device 100 (e.g., the waist support frame 20, the base body 80, or the leg driving frames 50 and 55). In an embodiment, the motion information of the components of the wearable device 100 may correspond to body motion information of a user. The sensor module 520 may transmit the obtained sensor data to the processor 512 or store the obtained sensor data in a separate storage module (not shown) including the memory 514. The sensor module 520 may include an IMU 522 and an angle sensor (e.g., the first angle sensor 524 and the second angle sensor 524-1) as shown in FIG. 5B. The IMU 522 (e.g., the IMU 135) may measure an upper body motion value of the user wearing the wearable device 100. For example, the IMU 522 may sense X-axis, Y-axis, and Z-axis accelerations and X-axis, Y-axis, and Z-axis angular velocities according to a motion of the user. The IMU 522 may be used to measure, for example, at least one of a forward and backward tilt, a left and right tilt, or a rotation of the body of the user. In an embodiment, the IMU 522 may obtain a motion value (e.g., an acceleration value and an angular velocity value) of a waist support frame (e.g., the waist support frame 20 of FIG. 3) or a base body (e.g., the base body 80 of FIG. 3) of the wearable device. The motion values of the waist support frame or the base body may correspond to the upper body motion values of the user.

The angle sensor (e.g., the angle sensor 125) may measure a hip joint angle value according to the leg motion of the user. Sensor data that may be measured by the angle sensor may include, for example, a hip joint angle value of a right leg, a hip joint angle value of a left leg, and information on a direction of a motion of a leg. For example, the first angle sensor 524 of FIG. 5B may obtain the hip joint angle value of the right leg of the user, and the second angle sensor 524-1 may obtain the hip joint angle value of the left leg of the user. The first angle sensor 524 and the second angle sensor 524-1 may each include, for example, an encoder and/or a Hall sensor. Further, the angle sensor may obtain a motion value of the leg driving frame of the wearable device 100. For example, the first angle sensor 524 may obtain a motion value of the first leg driving frame 55 of FIG. 3 and the second angle sensor 524-1 may obtain a motion value of the second leg driving frame 50. The motion values of the leg driving frame may correspond to the hip joint angle value of the user.

In an embodiment, the sensor module 520 may further include at least one of a position sensor configured to obtain a position value of the wearable device 100, a proximity sensor configured to sense the proximity of an object, a biosignal sensor configured to detect a biosignal of the user, or a temperature sensor configured to measure an ambient temperature. The types of sensors that may be included in the sensor module 520 are not limited to the examples described above.

The input module 540 may receive a command or data to be used by a component (e.g., the processor 512) of the wearable device 100 from the outside (e.g., a user) of the wearable device 100. The input module 540 may include an input component circuit. The input module 540 may include, for example, a key (e.g., a button) or a touch screen.

The sound output module 550 may output a sound signal to the outside of the wearable device 100. The sound output module 550 may provide auditory feedback to the user. For example, the sound output module 550 may include a speaker configured to play back a guiding sound signal (e.g., a driving start sound, an operation error notification sound, or an exercise start alarm), music content, or a guide voice for auditorily informing predetermined information (e.g., exercise result information or exercise posture evaluation information).

The haptic module 560 may provide haptic feedback to the user under the control of the processor 512. The haptic module 560 may convert an electrical signal into a mechanical stimulus (e.g., a vibration or a movement) or an electrical stimulus, which may be recognized by a user via his or her tactile sensation or kinesthetic sensation. The haptic module 560 may include a motor, a piezoelectric element, or an electrical stimulation device. In an embodiment, the haptic module 560 may be positioned in at least one of the base body (e.g., the base body 80) or a thigh fastening portion (e.g., the first thigh fastening portion 2 or the second thigh fastening portion 1).

In an embodiment, the control systems 500 and 500-1 may include a battery (not shown) for supplying power to each component of the wearable device 100 and a power management circuit (not shown) for converting the power of the battery into an operating voltage of each component of the wearable device 100 and supplying the converted power to each component.

The driving module 530 may generate an external force to be applied to the legs of the user under the control of the processor 512. The driving module 530 may generate torque to be applied to the legs of the user based on a control signal generated by the processor 512. The processor 512 may transmit a control signal for controlling the operation of the motor 534 to the motor driver circuit 532. The motor driver circuit 532 may control the operation of the motor 534 by generating a current signal (or voltage signal) corresponding to the control signal received from the processor 512 and supplying the generated current signal to the motor 534. The current signal may not be supplied to the motor 534 depending on an operation mode of the wearable device 100. When the motor 534 is supplied with the current signal and driven, the motor 534 may generate torque for an assistance force for assisting a leg motion of the user or a resistance force for hindering a leg motion of the user.

The processor 512 may execute software to control at least another component (e.g., a hardware or software component) of the wearable device connected to the processor 512 and may perform various types of data processing or operations. For example, the processor 512 may generate a control signal to control each component (e.g., the communication module 516, the driving module 530, the sound output module 550, or the haptic module 560) of the wearable device 100. The software executed by the processor 512 may include an application for providing a GUI. According to an embodiment, as at least a part of data processing or computation, the processor 512 may store instructions or data received from another component (e.g., the communication module 516) in the memory 514, process the instructions or data stored in the memory 514, and store result data obtained after processing in the memory 514. According to an embodiment, the processor 512 may include a main processor (e.g., a central processing unit (CPU) or an application processor (AP)) or an auxiliary processor (e.g., a graphics processing unit (GPU), a neural processing unit (NPU), an image signal processor (ISP), a sensor hub processor, or a communication processor (CP)) that is operable independently of or in conjunction with the main processor. The auxiliary processor may be implemented separately from the main processor or as a part of the main processor.

The memory 514 may store various pieces of data used by at least one component (e.g., the processor 512) of the wearable device 100. The data may include, for example, software, sensor data, and input data or output data for instructions related thereto. The memory 514 may include a volatile memory or a non-volatile memory (e.g., random-access memory (RAM), dynamic RAM (DRAM), or static RAM (SRAM)).

The communication module 516 may support establishing a direct (e.g., wired) communication channel or a wireless communication channel between the processor 512 and another component of the wearable device 100 or an external electronic device (e.g., the electronic device 210 or the other wearable device 220) and performing communication via the established communication channel. The communication module 516 may include a communication circuit configured to perform a communication function. For example, the communication module 516 may receive a control signal from an electronic device (e.g., the electronic device 210) and transmit the sensor data obtained by the sensor module 520 to the electronic device. According to an embodiment, the communication module 516 may include one or more CPs (not shown) that are operable independently of the processor 512 and that support direct (e.g., wired) communication or wireless communication. According to an embodiment, the communication module 516 may include a wireless communication module (e.g., a cellular communication module, a short-range wireless communication module, or a global navigation satellite system (GNSS) communication module), and/or a wired communication module. A corresponding one of the above communication modules may communicate with another component of the wearable device 100 and/or an electronic device via a short-range communication network, such as Bluetooth™, Wi-Fi, or infrared data association (IrDA), or a long-range communication network, such as a legacy cellular network, a 5G network, a next-generation communication network, the Internet, or a computer network (e.g., a local area network (LAN) or a wide region network (WAN)).

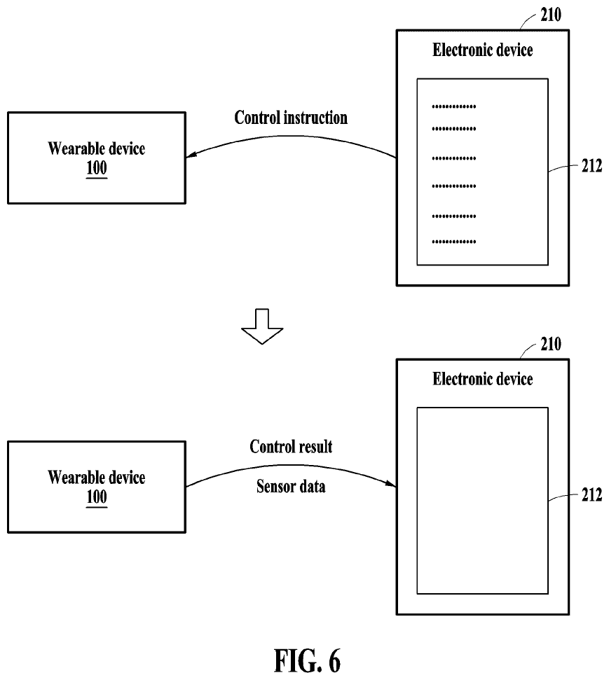

FIG. 6 is a diagram illustrating an interaction between a wearable device and an electronic device according to an embodiment.

Referring to FIG. 6, the wearable device 100 may communicate with the electronic device 210. For example, the electronic device 210 may be a user terminal of a user who uses the wearable device 100 or a controller device dedicated to the wearable device 100. In an embodiment, the wearable device 100 and the electronic device 210 may be connected to each other via short-range wireless communication (e.g., Bluetooth™ or Wi-Fi communication).

In an embodiment, the electronic device 210 may check a state of the wearable device 100 or execute an application to control or operate the wearable device 100. A screen of a user interface (UI) may be displayed to control an operation of the wearable device 100 or determine an operation mode of the wearable device 100 on a display 212 of the electronic device 210 through the execution of the application. The UI may be, for example, a GUI.

In an embodiment, the user may input an instruction for controlling the operation of the wearable device 100 (e.g., an execution instruction to a walking assistance mode, an exercise assistance mode, or a physical ability measurement mode) or change settings of the wearable device 100 through a GUI screen on the display 212 of the electronic device 210. The electronic device 210 may generate a control instruction (or control signal) corresponding to an operation control instruction or a setting change instruction input by the user and transmit the generated control instruction to the wearable device 100. The wearable device 100 may operate according to the received control instruction and transmit a control result according to the control instruction and/or sensor data measured by the sensor module of the wearable device 100 to the electronic device 210. The electronic device 210 may provide the user with result information (e.g., walking ability information, exercise ability information, or exercise result information) derived by analyzing the control result and/or the sensor data through the GUI screen.

In an embodiment, interaction between the wearable device 100 and the electronic device 210 may be performed under the control of the wearable device 100. The wearable device 100 may perform an operation actively rather than passively through a request or control from the electronic device 210. For example, when the connection with the electronic device 210 is initially detected, the wearable device 100 may check whether the user is properly wearing the wearable device 100 even without the control of the electronic device 210, and transmit a result of the check to the electronic device 210. In addition, when the proper wearing of the wearable device 100 is checked, the wearable device 100 may perform a sensor initialization process of initializing sensor data output from a sensor to accurately measure motion information of the user even without the control of the electronic device 210. When the sensor initialization process is completed, the wearable device 100 may notify the electronic device 210 of the completion of the sensor initialization by transmitting notification data.

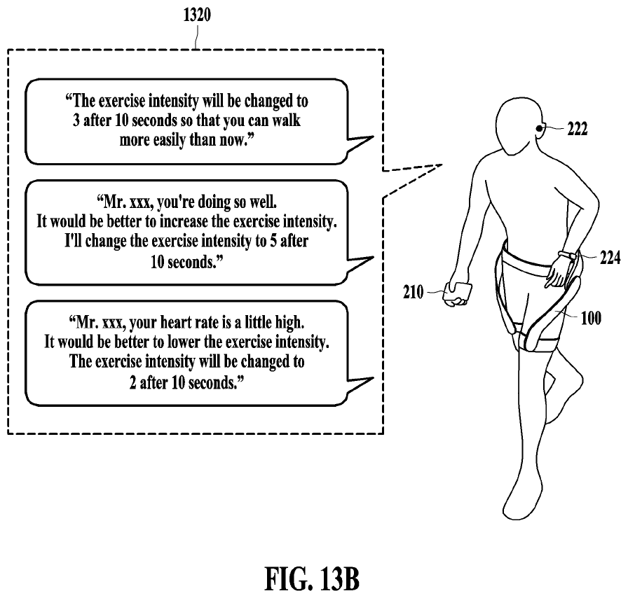

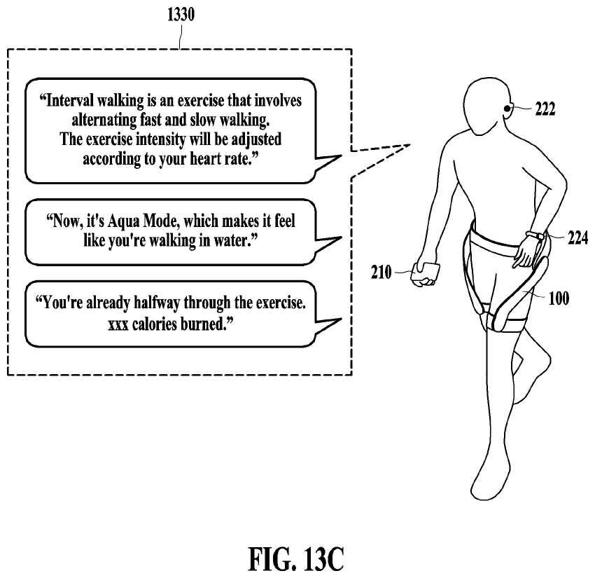

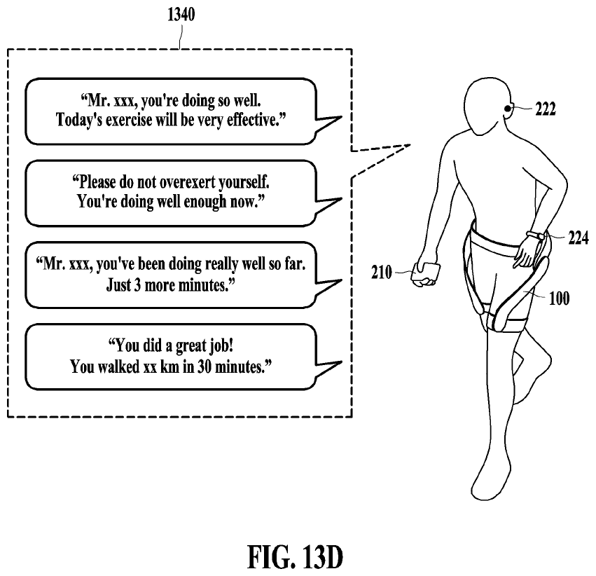

In an embodiment, the wearable device 100 and the electronic device 210 may interact to reduce the dependency on an application executed on the electronic device 210 when performing exercise assistance for the user. For example, the wearable device 100 and the electronic device 210 may reduce the dependency on the application through guidance via a guide voice and decision making using voice recognition and/or gesture recognition. If a series of manipulations (e.g., selecting an exercise program, setting exercise intensity, and the like) must be performed on a screen of the electronic device 210 through an application of the electronic device 210 when the user tries to start exercising using the wearable device 210, the usability may be reduced because the manipulation must be performed while looking at the screen. However, the wearable device 100 and the electronic device 210 may reduce the dependency on the application by suggesting an exercise program to be recommended to the user through a guide voice based on personalized data of the user and using voice recognition and/or gesture recognition to reduce manipulation on the screen of the electronic device 210. In addition, the wearable device 100 and the electronic device 210 may reduce the dependency on the application by performing exercise coaching in real time through voice feedback (e.g., guide voice) during the exercise of the user, and providing feedback information using other wearable devices (e.g., the wireless earphones 222 or the smartwatch 224). The guide voice for exercise coaching will be described in more detail with reference to FIG. 13A and subsequent drawings.

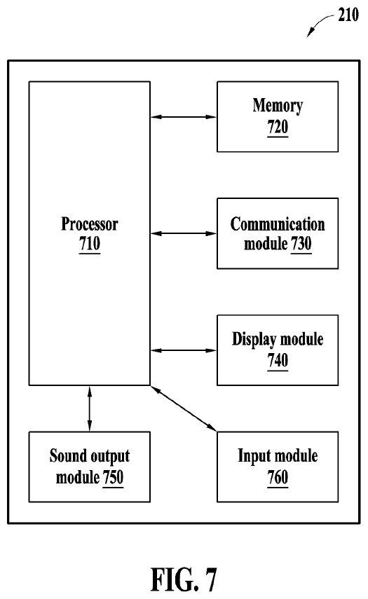

FIG. 7 is a diagram illustrating a configuration of an electronic device according to an embodiment.

Referring to FIG. 7, the electronic device 210 may include a processor 710, a memory 720, a communication module 730, a display module 740, a sound output module 750, and an input module 760. In an embodiment, at least one (e.g., the sound output module 750) of these components may be omitted from the electronic device 210, or one or more other components (e.g., a sensor module, a haptic module, and a battery) may be added to the electronic device 210.

The processor 710 may control at least one other component (e.g., a hardware or software component) of the electronic device 210, and may perform a variety of data processing or computation. According to an embodiment, as at least a part of data processing or computation, the processor 710 may store instructions or data received from another component (e.g., the communication module 730) in the memory 720, process the instructions or data stored in the memory 720, and store result data in the memory 720.

According to an embodiment, the processor 710 may include a main processor (e.g., a CPU or an AP) or an auxiliary processor (e.g., a GPU, an NPU, an ISP, a sensor hub processor, or a CP) that is operable independently of or in conjunction with the main processor.

The memory 720 may store various pieces of data used by at least one component (e.g., the processor 710 or the communication module 730) of the electronic device 210. The data may include, for example, a program (e.g., an application), and input data or output data for instructions related thereto. The memory 720 may include at least one instruction executable by the processor 710. The memory 720 may include, for example, a volatile memory or a non-volatile memory.

The communication module 730 may support establishing a direct (e.g., wired) communication channel or a wireless communication channel between the electronic device 210 and another electronic device (e.g., the wearable device 100, the other wearable device 220, or the server 230) and performing communication via the established communication channel. The communication module 730 may include a communication circuit configured to perform a communication function. The communication module 730 may include one or more CPs that are operable independently of the processor 710 (e.g., an AP) and that support a direct (e.g., wired) communication or a wireless communication. According to an embodiment, the communication module 730 may include a wireless communication module configured to perform wireless communication (e.g., a Bluetooth communication module, a cellular communication module, a Wi-Fi communication module, or a GNSS communication module) or a wired communication module (e.g., a LAN communication module or a power line communication module). For example, the communication module 730 may transmit a control instruction to the wearable device 100 and receive, from the wearable device 100, at least one of sensor data including body motion information of the user wearing the wearable device 100, state data of the wearable device 100, or control result data corresponding to the control instruction. The communication module 740 may transmit guide data and/or notification data to the other wearable device 220. The communication module 740 may transmit sensor data and user data received from the wearable device 100 to the server 230 and receive exercise result data and exercise program data from the server 230.

The display module 740 may visually provide information to the outside (e.g., a user) of the electronic device 210. The display module 740 may include, for example, a liquid-crystal display (LCD) or organic light-emitting diode (OLED) display, a hologram device, or a projector device. The display module 740 may further include a control circuit configured to control the driving of a display. In an embodiment, the display module 740 may further include a touch sensor set to sense a touch or a pressure sensor set to sense the intensity of a force generated by the touch. The display module 740 may output a UI screen for controlling the wearable device 100 or providing a variety of information (e.g., exercise evaluation information and setting information of the wearable device 100).

The sound output module 750 may output a sound signal to the outside of the electronic device 210. The sound output module 750 may include a guide sound signal (e.g., a driving start sound or an operation error notification sound) based on a state of the wearable device 100 and a speaker for playing musical content or a guide voice. When it is determined that the wearable device 100 is not properly worn on the body of the user, the sound output module 750 may output, for example, a guide voice for informing that the user is wearing the wearable device 100 abnormally or for guiding the user to wear the wearable device 100 properly. The sound output module 750 may output, for example, a guide voice corresponding to exercise evaluation information or exercise result information obtained by evaluating an exercise of the user.

The input module 760 may receive a command or data to be used by a component (e.g., the processor 710) of the wearable device 210 from the outside (e.g., a user) of the electronic device 210. The input module 760 may include an input component circuit and may receive a user input. The input module 760 may include, for example, a touch recognition circuit for recognizing a touch on a key (e.g., a button) and/or a screen.

In an embodiment, the electronic device 210 may include the communication module 730 configured to communicate with the wearable device 100, and the processor 710 configured to control an exercise of a user performed using the wearable device 100 based on the communication with the wearable device 100.

The processor 710 may output a first guide voice for guiding proper wearing of the wearable device 100 when connection to the wearable device 100 is detected. The communication module 730 may receive first notification data indicating that the user is properly wearing the wearable device 100 from the wearable device 100, and the processor 710 may output a second guide voice for guiding an operation for sensor initialization of the wearable device 100 in response to the reception of the first notification data. The sensor initialization process is a process corresponding to the initialization process for sensor data output by the sensor module 520 of the wearable device 100, which is a process of adjusting sensor data output from the sensor module 520 to a reference value when the user takes a ready posture to start exercising or measure physical ability. Since a body state in the ready posture (e.g., a leg angle or a body tilt) may be different for each user, the process of initializing the sensor data output from the sensor module 520 of the wearable device 100 in the ready posture of the user may be required to accurately measure a motion and/or posture of the user.

The communication module 730 may receive second notification data indicating that the sensor initialization is completed from the wearable device 100, and the processor 710 may output a third guide voice for sequentially inquiring whether to select each one of a plurality of recommended exercise programs in response to the reception of the second notification data. In an embodiment, the processor 710 may determine recommended exercise programs based on the personalized data of the user and situation data. The personalized data may include, for example, exercise ability indicator information of the user (e.g., muscle strength, cardiovascular endurance, flexibility, symmetry, or core stability), exercise amount information (e.g., the amount of exercise of this week or the amount of exercise of this month), and physical information of the user (e.g., a height, weight, or BMI). The situation data may include, for example, time (e.g., including day of the week and a season) and information about the weather.

In an embodiment, the processor 710 may output a third guide voice for inquiring whether to select a next recommended exercise program in at least one of a case where motion data of the user is not received from the wearable device 100 and a case where a gesture motion corresponding to refusal of a current recommended exercise program is recognized during a predefined period of time (e.g., 3 seconds) after the third guide voice for inquiring selection of the current recommended exercise program is output.

In an embodiment, the processor 710 may output a third guide voice for inquiring selection of a first recommended exercise program among the recommended exercise programs, and output a third guide voice for inquiring selection of a second recommended exercise program among the recommended exercise programs when response data corresponding to the selection of the first recommended exercise program is not received during a predefined period of time. The processor 710 may output a third guide voice for inquiring selection of a third recommended exercise program among the recommended exercise programs when response data corresponding to the selection of the second recommended exercise program is not received during a predefined period of time.

In an embodiment, the communication module 730 may communicate with the wireless earphones 222, and the first guide voice, the second guide voice, and the third guide voice may be output through the sound output module 750 and/or the wireless earphones 222. In an embodiment, the processor 710 may activate a voice feedback function for outputting the first guide voice, the second guide voice, and the third guide voice when a currently set audio volume value of the electronic device 210 is greater than or equal to a reference value. The processor 710 may inactivate the voice feedback function when the currently set audio volume value is less than the reference value or 0. When the voice feedback feature is inactivated, guide information corresponding to each of the first guide voice, the second guide voice, and the third guide voice may be provided on a screen through the display module 740.

In an embodiment, the communication module 730 may receive response data corresponding to selection of a target recommended exercise program among the recommended exercise programs from the wearable device 100, and the processor 710 may execute an exercise mode according to the target recommended exercise program in response to the reception of the response data. The processor 710 may determine whether the user wearing the wearable device 100 has taken a gesture (e.g., a walking-in-place posture) corresponding to the selection of the target recommended exercise program based on motion data of the user received from the wearable device 100. The processor 710 may determine a recommended exercise program that is finally guided through the third guide voice as the target recommended exercise program when it is determined that the gesture motion is performed. In an embodiment, a gesture motion in which the user is standing still for a predetermined period of time without moving his/her legs or other active gesture motions (e.g., raising or lowering only the right or left leg) of the user wearing the wearable device 100 may be recognized as a response of refusal of the selection.

In an embodiment, the processor 710 may output a list of other recommended exercise programs through the display module 740 when the response data corresponding to the selection of the target recommended exercise program is not received until an output of a third guide voice for sequentially inquiring whether to select each of the recommended exercise programs is completed. The user may select a target exercise program to perform from the list of recommended exercise programs displayed on the screen of the display module 740. The processor 710 may perform an exercise mode according to the selected target exercise program.

In an embodiment, the processor 710 may provide the user with a guide voice for exercise coaching/guidance based on the exercise situation and the physical condition (e.g., a heart rate) of the user during the exercise of the user. For example, the processor 710 may provide a guide voice related to real-time guidance, a change of an exercise type/exercise intensity, guidance, providing of information, and encouragement to improve the exercise effect of the user. The guide voice may be provided to the user through the wireless earphones 222 connected to the sound output module 750 or the electronic device 210.

While the user performs the exercise according to the target exercise program selected by the user, the wearable device 100 may transmit motion data of the user to the electronic device 210. The processor 710 may determine whether an exercise state is a stop of an exercise based on the motion data of the user wearing the wearable device 100 received through the communication module 730. For example, based on the motion data of the user, when it is recognized that the user has taken a posture different from the exercise postures of the target exercise program, a stop posture for a predetermined period of time, or a predefined exercise stop gesture motion (e.g., walking in place), the processor 710 may determine the exercise state of the user as the stop of the exercise. When the exercise state is determined as the stop of the exercise, the processor 710 may output a fourth guide voice for inquiring whether to terminate the exercise to the user.

In an embodiment, the communication module 730 may receive response data corresponding to selection of the stop of the exercise from the wearable device 100, and the processor 710 may provide the user with exercise result data about the exercise performed by the user in response to the reception of the response data. When the user wants to select the stop of the exercise, the user may perform, for example, a gesture motion of walking in place while wearing the wearable device 100. The processor 710 may determine whether the user wearing the wearable device 100 has performed a gesture motion corresponding to the selection of the stop of the exercise based on the motion data of the user received from the wearable device 100. When it is determined that the gesture motion has been performed, the processor 710 may determine that the exercise of the user is completed. The processor 710 may provide the user with exercise result data through an audio signal. The processor 710 may output a fifth guide voice for guiding to take the wearable device 100 off after providing the user with the exercise result data. The fourth guide voice and the fifth guide voice may be output through the sound output module 750 and/or the wireless earphones 222.

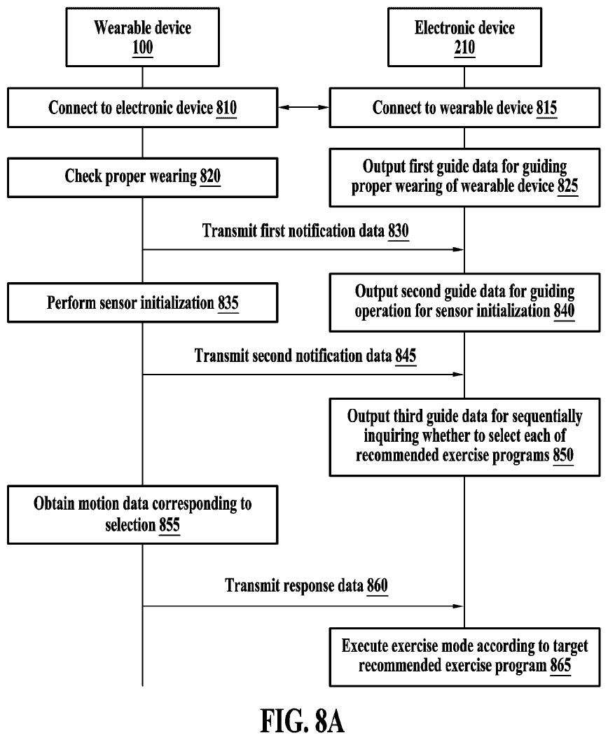

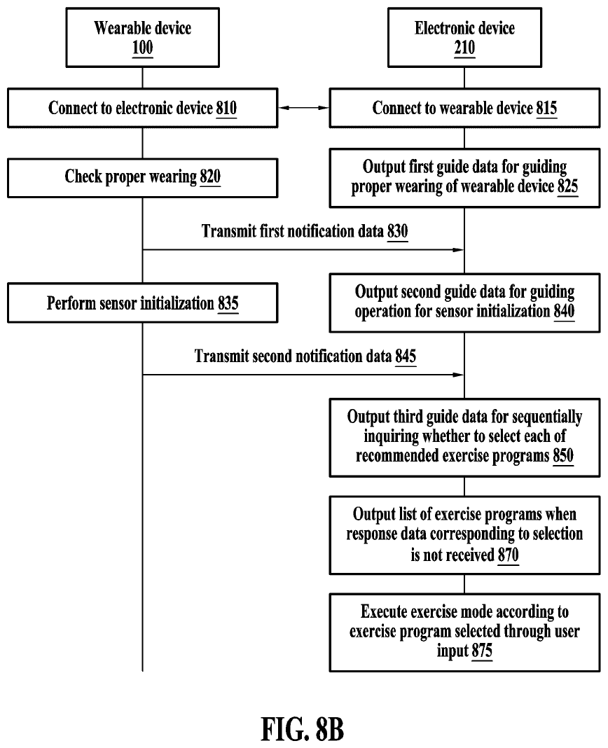

FIGS. 8A and 8B are flowcharts illustrating interaction methods between a wearable device and an electronic device in an exercise preparation and exercise starting operation according to an embodiment. In an embodiment, at least one of operations of FIGS. 8A and 8B may be performed simultaneously or in parallel with another operation, and the order of the operations may be changed. In addition, some of the operations may be omitted or another operation may be additionally performed.

Referring to FIG. 8A, the wearable device 100 may perform connection (e.g., connection via wireless communication) to the electronic device 210 in operation 810, and the electronic device 210 may perform connection to the wearable device in operation 815. In an embodiment, when a motion of the wearable device 100 (e.g., a motion of a leg driving frame and/or waist support frame) is detected in a standby mode, the wearable device 100 may switch an operation mode from the standby mode to a normal mode. The normal mode may be a mode in which the wearable device 100 may be used normally, and the standby mode may be a mode in which only essential activities of components are activated to reduce power consumption of the wearable device 100. The wearable device 100 may automatically search for the electronic device 210 to attempt the connection from the surroundings of the wearable device 100 in the normal mode.

When the connection to the electronic device 210 is completed, in operation 820, the wearable device 100 may check whether the user is wearing the wearable device 100 properly. In operation 825, when the connection to the wearable device 100 is detected, the electronic device 210 may output first guide data (e.g., a first guide voice) for guiding the proper wearing of the wearable device 100.

When it is determined that the user is properly wearing the wearable device 100, in operation 830, the wearable device 100 may transmit first notification data indicating that the user is properly wearing the wearable device 100 to the electronic device 210. The electronic device 210 may receive the first notification data from the wearable device 100, and output second guide data (e.g., a second guide voice) for guiding an operation for sensor initialization of the wearable device 100 in operation 840 in response to the reception of the first notification data. In operation 835, the wearable device 100 may perform the sensor initialization. The wearable device 100 may perform the sensor initialization of setting sensor data output from a sensor module as a reference value during a time section, in which the second guide data is output. During the time section, the user may take a ready posture for the sensor initialization according to the second guide data.

When the sensor initialization is completed, in operation 845, the wearable device 100 may transmit second notification data indicating that the sensor initialization is completed to the electronic device 210. The electronic device 210 may receive the second notification data from the wearable device 100, and output third guide data (e.g., a third guide voice) for sequentially inquiring whether to select each of a plurality of recommended exercise programs in operation 850 in response to the reception of the second notification data. The electronic device 210 may select recommended exercise programs optimized for the user based on personalized data of the user, and suggest the recommended exercise programs to the user. The personalized data and the recommended exercise programs may be transmitted from a server (e.g., the server 230 of FIG. 2) connected to the electronic device 210. For example, the recommended exercise programs may include walking programs with various times, walking speeds, levels of intensity required when walking, walking courses.

In an embodiment, the electronic device 210 may output the third guide voice for inquiring whether to select a next recommended exercise program in at least one of a case where motion data of the user is not received from the wearable device 100 and a case where a gesture motion corresponding to refusal of the current recommended exercise program is recognized during a predefined period of time after the third guide voice for inquiring selection of the current recommended exercise program is output.

In an embodiment, the electronic device 210 may output a third guide voice for inquiring selection of a first recommended exercise program among the recommended exercise programs, and output a third guide voice for inquiring selection of a second recommended exercise program among the recommended exercise programs when response data corresponding to the selection of the first recommended exercise program is not received during a predefined period of time. The electronic device 210 may output a third guide voice for inquiring selection of a third recommended exercise program among the recommended exercise programs when response data corresponding to the selection of the second recommended exercise program is not received during a predefined period of time.

While the recommended exercise programs are sequentially guided through the third guide voice, in operation 855, the wearable device 100 may obtain motion data corresponding to the selection according to the gesture motion of the user. The user may perform a gesture motion corresponding to the selection within the time section in which the target recommended exercise program to select is guided among the recommended exercise programs sequentially guided through the third guide voice. For example, the gesture motion corresponding to the selection may be a walking-in-place posture. In an embodiment, the electronic device 210 may also receive a selection input for the target recommended exercise program from the user through voice recognition.

In operation 860, the wearable device 100 may transmit response data including the motion data to the electronic device 210. In operation 865, when the electronic device 210 receives the response data corresponding to the selection of the target recommended exercise program among the recommended exercise programs from the wearable device 100, the electronic device 210 may execute an exercise mode according to the target recommended exercise program. In an embodiment, the electronic device 210 may determine whether the user wearing the wearable device 100 has performed the gesture motion corresponding to the selection of the target recommended exercise program based on the motion data of the user received from the wearable device 100. When it is determined that the gesture motion has been performed, the electronic device 210 may determine a recommended exercise program that is finally guided through the third guide voice as the target recommended exercise program.

In an embodiment, when it is determined that the user has performed the gesture motion corresponding to the selection of the target recommended exercise program, the electronic device 210 may output the guide data for inquiring whether the user actually selected the target recommended exercise program. When motion data corresponding to the gesture motion induced through the guide data is received from the wearable device 100 during a predefined period of time after the guide data is output, the electronic device 210 may determine that the user actually selected the target recommended exercise program and execute an exercise mode according to the target recommended exercise program. When the motion data corresponding to the induced gesture motion is not received from the wearable device 100, the electronic device 210 may proceed to a next step without determining the target recommended exercise program.

Referring to FIG. 8B, operations 810 to 850 are the same as operations 810 to 850 of FIG. 8A, and thus any repeated description is omitted. While the third guide data is output in operation 850, the user may refuse all selections of the recommended exercise programs guided through the third guide data. In an embodiment, the user may express intention of the refusal of selection to the electronic device 210 by not making any movement while wearing the wearable device 100 or by taking a gesture motion corresponding to the refusal of selection. In this embodiment, it is assumed that the user has refused to select any of the recommended exercise programs guided through the third guide data because the user has not performed any motion. For example, the user may not perform a gesture motion corresponding to the selection of the target recommended exercise program while three recommended exercise programs are guided sequentially through the third guide voice.

In operation 870, when the response data corresponding to the selection of the target recommended exercise program is not received until an output of the third guide voice for sequentially inquiring whether to select each of the recommended exercise programs is completed, the electronic device 210 may output a list of exercise programs through the screen. The user may select an exercise program to perform from the output list of the exercise programs through a user input. The user may adjust an exercise option (e.g., an exercise time, exercise difficulty, or exercise course) for the exercise program to perform through an application executed on the electronic device 210. In operation 875, the electronic the device 210 may execute the exercise mode according to the exercise program selected through the user input.

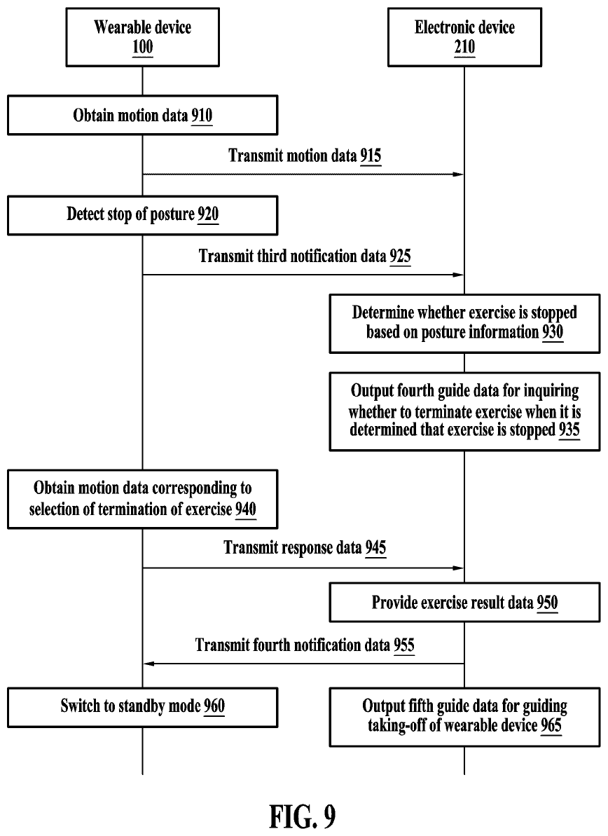

FIG. 9 is a flowchart illustrating an interaction method between a wearable device and an electronic device in an exercise progress and exercise completion operation according to an embodiment. In an embodiment, at least one of operations of FIG. 9 may be performed simultaneously or in parallel with another operation, and the order of the operations may be changed. In addition, some of the operations may be omitted or another operation may be additionally performed.

Referring to FIG. 9, in operation 910, the wearable device 100 may obtain motion data of the user during the exercise through a sensor module. In operation 915, the wearable device 100 may transmit the obtained motion data to the electronic device 210. The electronic device 210 may analyze the exercise of the user based on the obtained motion data or transmit the motion data to another device (e.g., the server 230). The electronic device 210 may perform exercise coaching to the user in real time based on a result of the exercise analysis. Feedback information for the exercise coaching may be provided through a sound output model of the electronic device 210 and/or the wireless earphones 222 connected to the electronic device 210.

In operation 920, the wearable device 100 may detect a stop of posture of the user based on the motion data of the user during the exercise. For example, when there is no change in motion data for a predetermined period of time, the wearable device 100 may detect that the stop of the posture has occurred. When the stop of the posture is detected, in operation 925, the wearable device 100 may transmit third notification data indicating that the stop of the posture is detected to the electronic device 210.

In operation 930, the electronic device 210 may determine whether the exercise is stopped based on the motion data of the user wearing the wearable device 100. The electronic device 210 may determine whether the user intends to stop exercising or to take a break during exercising based on the motion data. The electronic device 210 may estimate a current posture of the user based on the motion data, and determine whether the user intends to stop exercising or take a break according to the estimated current posture. In operation 935, when the exercise state is determined as the stop of the exercise, the electronic device 210 may output fourth guide data (e.g., a fourth guide voice) for inquiring whether to terminate the exercise to the user. When it is determined that the user is taking a break according to the current posture, the fourth guide data may not be output.

The user may select whether to terminate the exercise in response to the output of the fourth guide data. In operation 940, the wearable device 100 may obtain the motion data of the user corresponding to the selection of the termination of the exercise. For example, the user may perform a gesture motion (e.g., walking-in-place) corresponding to the selection of the termination of the exercise to select the termination of the exercise, and the wearable device 100 may obtain motion data corresponding to the gesture motion. In operation 945, the wearable device 100 may transmit the response data including the motion data to the electronic device 210.

In operation 950, the electronic device 210 may provide the user with exercise result data for the exercise performed by the user in response to the reception of the response data corresponding to the selection of the stop of the exercise from the wearable device 100. When the response data is received, the electronic device 210 may terminate the execution of the exercise program and analyze result information about the exercise performed by the user up to a current stage to provide the user with a result of the analysis of the exercise performance through a voice or on a screen. In an embodiment, the motion data obtained during the exercise of the user may be transmitted to the server 230, and when the termination of the exercise is determined, the server 230 may transmit the result of the analysis of the exercise performance of the user analyzed based on the motion data to the electronic device 210. The electronic device 210 may provide the user with the result of the analysis of the exercise performance received from the server 230.

After the exercise result data is provided, in operation 955, the electronic device 210 may transmit fourth notification data indicating that the providing of the exercise result data is completed to the wearable device 100. In operation 960, the wearable device 100 may switch the operation mode from the normal mode to the standby mode in response to the reception of the fourth notification data.

In operation 965, the electronic device 210 may output fifth guide data (e.g., a fifth guide voice) for guiding taking-off of the wearable device 100 after the exercise result data is provided to the user.

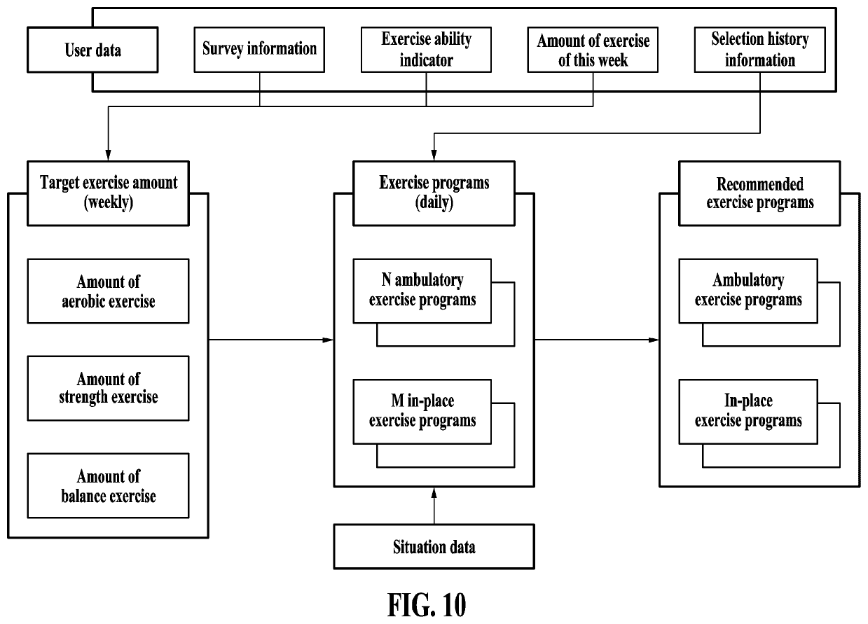

FIG. 10 is a diagram illustrating determination of a recommended exercise program to be provided to a user according to an embodiment.

Referring to FIG. 10, the electronic device 210 may select recommended exercise programs based on personalized data of the user and suggest the recommended exercise programs to the user, in order to reduce the dependency on an application when the user selects an exercise program using the wearable device 100. In an embodiment, selection of the recommended exercise programs for a user to be described below may be performed by the server 230. The server 230 may transmit information about the selected recommended exercise programs to the electronic device 210.

In an embodiment, the electronic device 210 may obtain survey information, exercise ability indicators, the amount of exercise of this week (or the amount of exercise of the last week), and selection history information as user data. The survey information may include, for example, age, gender, height, weight, exercise goal, physical fitness level, medical history, current medical condition, and BMI. The exercise ability indicators may include information about muscle strength, cardiovascular endurance, core stability, flexibility, and symmetry indicating the exercise abilities of the user. The amount of exercise of this week may indicate the amount of exercise performed by the user this week using the wearable device 100. The selection history information may indicate history information about the selection of the recommended exercise programs by the user.

In an embodiment, the electronic device 210 may determine a target exercise amount (e.g., weekly target exercise amount) for the user based on the user data. For example, the electronic device 210 may determine a weekly target value for each of the amount of strength exercise, the amount of aerobic exercise, and the amount of balance exercise.

In an embodiment, the electronic device 210 may select (or generate) a plurality of exercise programs in the units of days based on the target exercise amount, the selection history information, and situation data. The situation data may include, for example, time (e.g., including day of the week and the season) and information about the weather. The exercise programs may include, for example, ambulatory exercise programs (e.g., walking and running) and in-place exercise programs (e.g., fitness and stretching).

The electronic device 210 may select recommended exercise programs among exercise programs to minimize a difference between the target exercise amount and an actual amount of exercise performed so far. For example, when the user has done a lot of aerobic exercise up until yesterday but lacks strength and balance exercises, the electronic device 210 may select exercise programs with a relatively low proportion of the aerobic exercise and a relatively high proportion of the strength and balance exercise as the recommended exercise programs. For example, an exercise program that may achieve a daily target exercise amount may be selected as a recommended exercise program. The electronic device 210 may generate one or more recommended exercise programs by considering the exercise type (e.g., ambulatory exercise program or in-place exercise program), exercise time, and exercise effect according to the situation of the user, and suggest the one or more generated recommended exercise programs to the user. The recommended exercise programs may be suggested to the user through a guide voice or a UI screen of the electronic device 210 in an exercise starting operation.

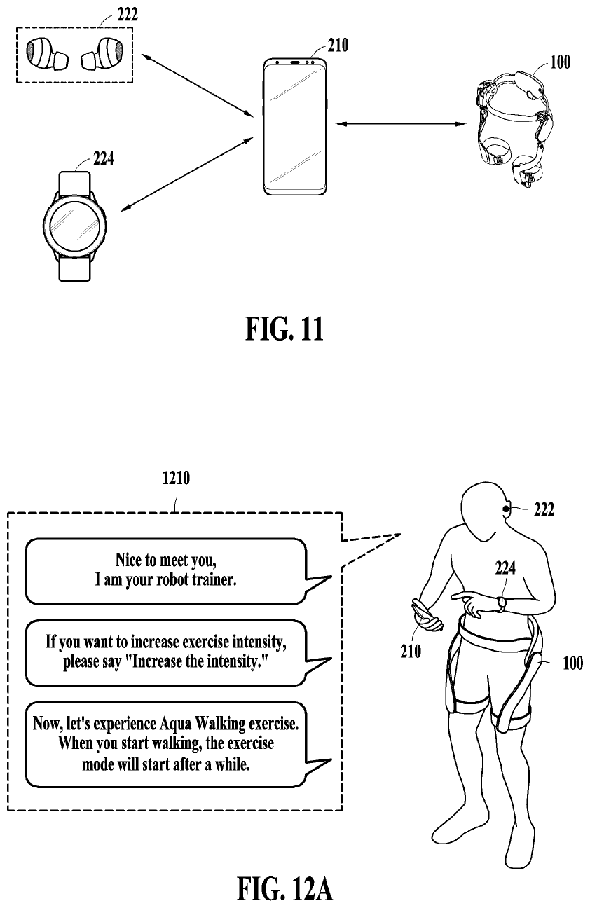

FIG. 11 is a diagram illustrating decision making in a multi-device environment according to an embodiment.

Referring to FIG. 11, the multi-device environment according to an embodiment may include the wearable device 100, the electronic device 210, the wireless earphones 222, and the smartwatch 224. The electronic device 210 may check the type of a device connected to the electronic device 210, and perform a control operation through interface means optimized for the connected device.

In an embodiment, when the electronic device 210 is connected to the wireless earphones 222, voice feedback may be provided to the user through the wireless earphones 222. For example, the electronic device 210 may output a guide voice through the wireless earphones 222. The electronic device 210 may activate a voice feedback function through the wireless earphones 222 when the electronic device 210 is connected to the wireless earphones 222. The electronic device 210 may also perform voice recognition based on a voice signal input through a microphone embedded in the wireless earphones 222.

In an embodiment, when the electronic device 210 is not connected to the wireless earphones 222, the electronic device 210 may determine whether to use the voice feedback function based on a currently set audio volume value. For example, when the currently set audio volume value is greater than or equal to a reference value, the electronic device 210 may determine to use the voice feedback function. When the currently set audio volume value is less than the reference value or is 0, the electronic device 210 may determine not to use the voice feedback function.

In an embodiment, when the electronic device 210 is connected to the smartwatch 224, a UI may be provided to display guide information or notification information through a screen of the smartwatch 224. When the voice feedback function is inactivated, the electronic device 210 may receive a selection input of the user based on the UI provided through the screen of the smartwatch 224 or the UI provided through the screen of the electronic device 210. In addition, the electronic device 210 may perform the voice recognition based on a voice signal input through a microphone embedded in the smartwatch 224. In an embodiment, the electronic device 210 may also perform gesture recognition for decision making based on motion data measured using the smartwatch 224.

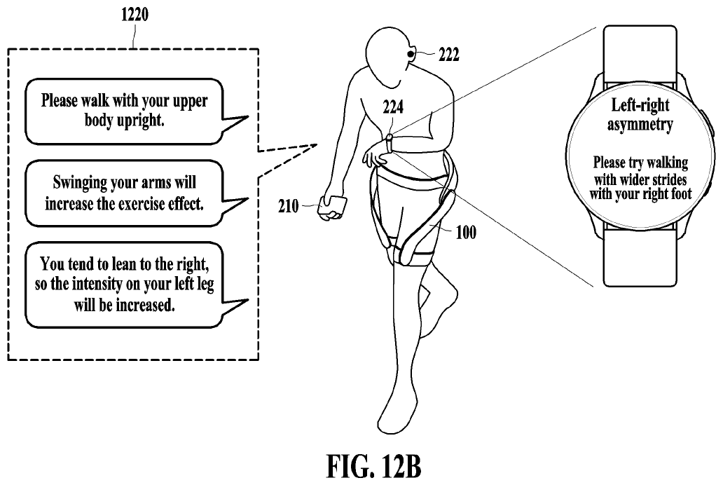

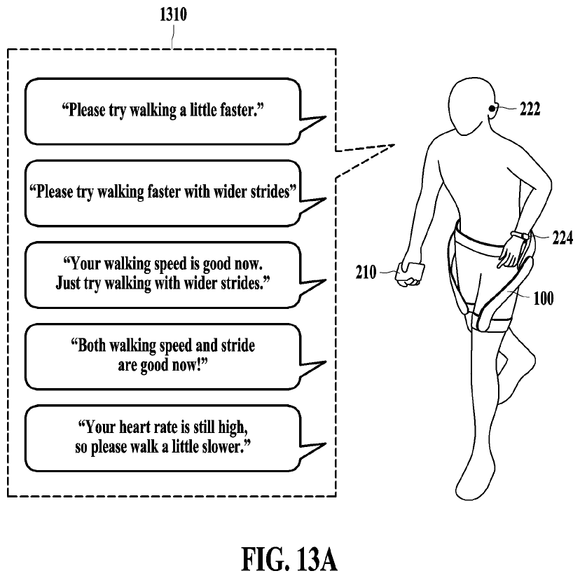

FIGS. 12A and 12B are diagrams illustrating examples in which guide voices are provided in a multi-device environment according to an embodiment.

Referring to FIG. 12A, a guide voice 1210 may be provided through voice feedback in the exercise preparation and exercise starting operation. For example, the wearable device 100 and the electronic device 210 may output a guide voice for guiding a control operation through the voice recognition and a guide voice for guiding the control operation to induce the start of an exercise mode. The electronic device 210 and the wearable device 100 may provide the user with a guide voice appropriate to a current situation based on a detected situation (e.g., detecting the power on of the wearable device 100, detecting the wearing of the wearable device 100 by the user, and detecting the need for the sensor initialization process). The user may perform the control operation through a voice dialogue using a voice recognition function of the electronic device 210. A guide voice may be output through the wireless earphones 222 and/or the sound output module of the electronic device 210.

Referring to FIG. 12B, in an exercise progress operation, the electronic device 210 may provide the user with a guide voice 1220 for real-time coaching/guidance, thereby improving the exercise effect of the user. The wearable device 100 may measure motion data of the user during the exercise of the user, and transmit the motion data to the electronic device 210 in real time, and the electronic device 210 may evaluate the exercise currently being performed by the user based on the motion data. For example, the electronic device 210 may determine whether an exercise posture and/or a motion speed of the user is appropriate, and provide a guide voice for more effective exercise. The evaluation of the exercise may be performed by a server (e.g., the server 230) connected to the electronic device 210. The guide voice may be output through the wireless earphones 222 and/or the sound output module of the electronic device 210. In an embodiment, guide data for real-time coaching may be output on the screen of the smartwatch 224. The user may check important exercise correction indicators in the current exercise situation through the screen of the smartwatch 224. In addition to the providing of the guide voice 1220, the electronic device 210 may also transmit a control instruction to the wearable device 100 during the exercise of the user to adjust exercise settings (e.g., exercise intensity and exercise time) for effective exercise performance.

FIGS. 13A, 13B, 13C, and 13D are diagrams illustrating a voice coaching/guidance function provided during an exercise process of a user according to an embodiment.

An exercise support system including the wearable device 100, the electronic device 210, and the wireless earphones 222 may perform a voice coaching function by providing the guide voice for the exercise (e.g., waking exercise) of the user. The voice coaching function may be implemented through a voice-based human-robot interaction (HRI), and may provide guide voices related to real-time guidance, a change of an exercise type/exercise intensity, guidance, providing of information, and encouragement to improve the exercise effect of the user. In some embodiments, the exercise support system may not include the wireless earphones 222. The exercise support system may be a subsystem of the exercise management system 200 of FIG. 2.

In an embodiment, when the wireless earphones 222 are worn on the user and connected to the electronic device 210 and/or the wearable device 100, the guide voice for the voice coaching may be output through the wireless earphones 222. When the wireless earphones 222 are not connected, the guide voice for the voice coaching function may be output through the electronic device 210 or the wearable device 100. The wireless earphones 222 may be connected to the electronic device 210 or the wearable device 100 in the one-to-one manner. In the exercise process of the user, sound effects for states of the wearable device 100, exercise states, changes, confirmations, and the like may be output through the wearable device 100, the electronic device 210, and/or the wireless earphones 222. In an embodiment, the output control of the guide voices and sound effects may be performed by the electronic device 210.

FIG. 13A illustrates examples 1310 of guide voices for real-time guide in a sensing-based voice coaching function according to an embodiment. The guide voices for real-time guide may be provided to the user periodically (e.g., once every 30 seconds). The electronic device 210 may determine whether a current walking speed of the user matches a target walking speed, whether a stride of the user is appropriate, or, whether a heart rate of the user is appropriate, and then provide the user with a guide voice according to a result of the determination. For example, when the current walking speed is lower than the target walking speed, a guide voice of “Please try walking a little faster” may be provided. When the current walking speed is lower than the target walking speed and the stride of the user is less than a reference stride, a guide voice of “Please try walking faster with wider strides” may be provided. When the current walking speed and strides are appropriate, a guide voice of “Both walking speed and stride are good now!” may be provided. When the heart rate of the user is continuously measured to be higher than a reference heart rate, a guide voice of “Your heart rate is still high, so please walk a little slower” may be provided.

FIG. 13B illustrates examples 1320 of guide voices for changing the exercise intensity in the sensing-based voice coaching function according to an embodiment. A guide voice for changing the exercise intensity may be provided to the user when the exercise sections are switched (e.g., when switched into a sprint section and a recovery section). A heart rate of the user measured in a previous exercise set section (e.g., the sprint section and the recovery section proceed sequentially) may be determined to what extent it satisfies a reference heart rate section by the electronic device 210, and a guide voice for changing the exercise intensity may be provided according to a result of the determination. As in the examples 1320 of the guide voice, when the measured heart rate of the user is continuously maintained to be higher than the reference heart rate section, a guide voice indicating to lower the exercise intensity in a next exercise set may be provided. When the measured heart rate of the user is continuously maintained to be lower than the reference heart rate section or when the measured heart rate of the user satisfies the reference heart rate section, a guide voice indicating to increase the exercise intensity in the next exercise set compared to the previous exercise set.

FIG. 13C illustrates examples 1330 of guide voices for guidance and information providing in the voice coaching function according to an embodiment. The guide voices for guidance and information providing may be provided to the user, for example, at an exercise start time point, a time point at which the exercise has reached 50% of an entire predicted exercise section, and an exercise termination time point. As in the examples 1330 of guide voices, when the start of exercise is detected, a guide voice for providing information about an exercise mode to be performed may be provided. When 50% of the exercise progress is detected, a guide voice for guiding that the exercise is halfway done and guiding calorie information burned during that time may be provided.

FIG. 13D illustrates examples 1340 of guide voices for encouragement (or motivation) in the voice coaching function according to an embodiment. The guide voices for encouragement may be provided to the user, for example, at a time point when entering a predefined exercise section. As in the examples 1350 of guide voices, a guide voice for encouraging the user to continue exercising, a guide voice for motivating the user to perform the exercise, and the like may be provided.

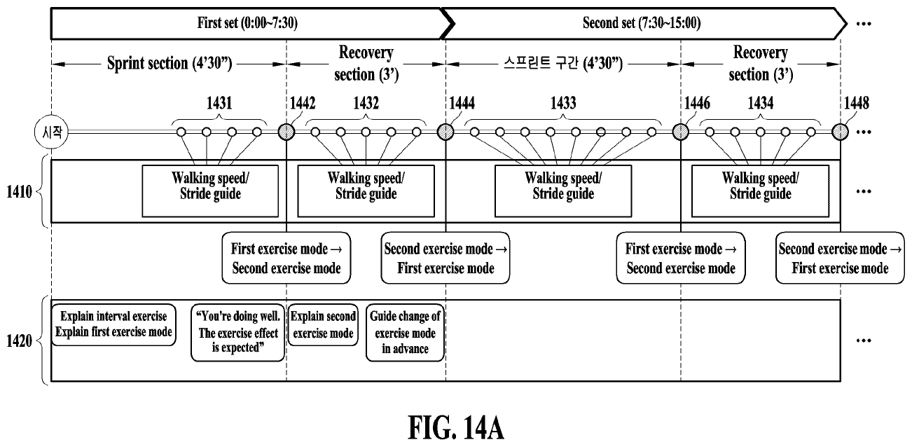

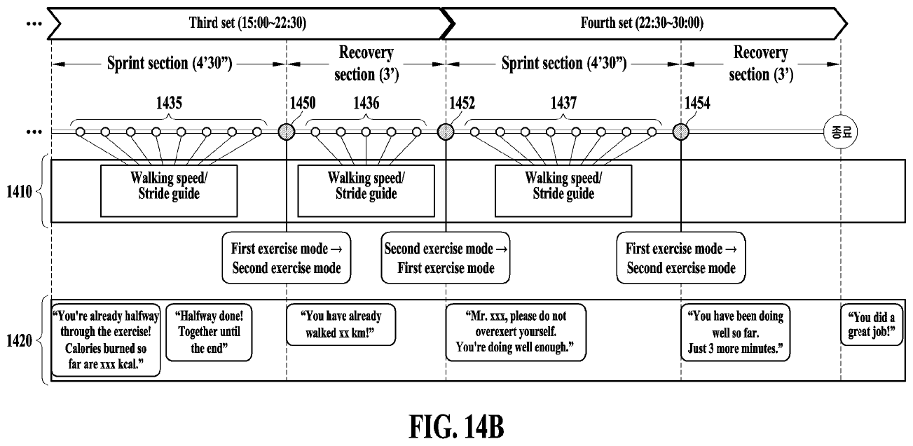

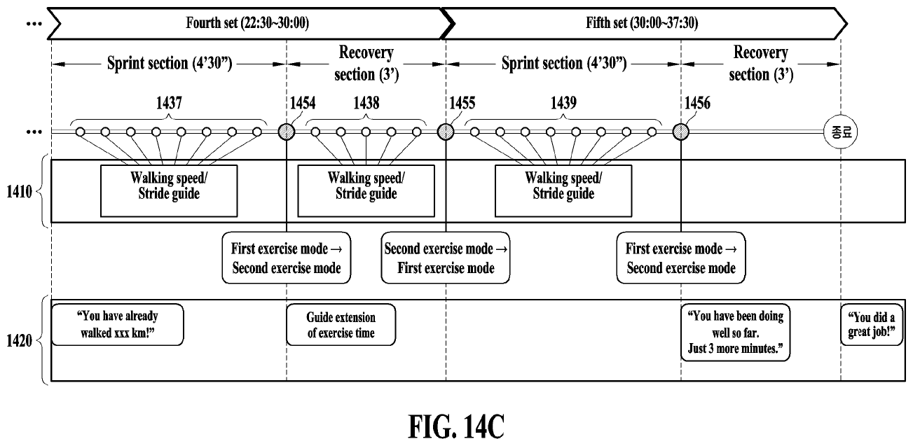

FIGS. 14A, 14B, and 14C are diagrams illustrating a voice coaching/guidance function provided for each set constituting an exercise program according to an embodiment.

FIGS. 14A, 14B, and 14C illustrate a case where the user selects to perform a 30-minute walking exercise including four sets before starting the exercise. The user may select the duration of the walking exercise to be performed (e.g., 15 minutes, 30 minutes, 45 minutes, or 60 minutes) before starting the exercise. Each set may include a sprint section of 4 minutes and 30 seconds and a recovery section of 3 minutes, however, is not limited thereto and various variations may be performed. A guide voice for the voice coaching function may be provided to the user in the walking exercise process of the user. A reference numeral 1410 may show guide voices provided in the sensing-based voice coaching function (e.g., real-time guide or change of the exercise intensity), and a reference numeral 1420 may show guide voices provided for guidance, providing of information, and encouragement. The heart rate of the user may be measured by a sensor included in the smartwatch 224 while the user is exercising, and the measured heart rate information may be transmitted to the electronic device 210. The electronic device 210 may determine whether the exercise intensity being applied to the user is appropriate, by comparing the heart rate of the user with the reference heart rate (or a heart rate zone or a heart rate range) in each set. For example, when the measured heart rate continues to exceed the reference heart rate, the exercise intensity may be determined to be high compared to the current physical condition of the user, and when the measured heart rate continues to be lower than the reference heart rate, the exercise intensity may be determined to be low compared to the current physical condition of the user.

Referring to FIG. 14A, when the user starts exercising and a first set proceeds, the explanation of an exercise program to be performed and the explanation of an exercise mode proceeding in the first set may be provided to the user through a guide voice. At each of exercise time points 1431, a guide voice for guiding a walking speed and/or strides of the user estimated based on sensor data of the wearable device 100 may be provided. At a time point 1442 at which the sprint section of the first set is completed, the exercise mode may be changed from a first exercise mode to a second exercise mode, and a guide voice for explaining the second exercise mode may be provided to the user. The guide voice for guiding the walking speed and/or strides of the user may be provided at each of exercise time points 1432 after the recovery section starts. At a time point 1444 at which the first set is completed, the exercise mode may be changed from the second exercise mode to the first exercise mode, and a guide voice for guiding that the exercise mode is to be changed may be provided before the change.

When the second set proceeds, the exercise intensity applied in the second set may be determined based on a result of comparing the heart rate of the user measured in the first set and the reference heart rate. When the heart rate measured in the first set continues to exceed the reference heart rate, the exercise intensity lower than the exercise intensity applied in the first set may be applied in the second set. When the heart rate measured in the first set continues to be lower than the reference heart rate, the exercise intensity higher than the exercise intensity applied in the first set may be applied in the second set. When the changed exercise intensity is applied, a guide voice for guiding the change of the exercise intensity may be provided.

At each of exercise time points 1433 in the sprint section and each of exercise time points 1434 in the recovery section in the second set, a guide voice for guiding the walking speed and/or strides of the user may be provided. At each of a time point 1446 at which the sprint section is completed and a time point 1448 at which the second set is completed, the exercise mode may be changed.

Referring to FIG. 14B, a third set may start after the second set is completed. The time point at which the second set is completed may correspond to a time point at which half of the entire exercise section is completed, and at this time, a guide voice for informing the user that the half of the exercise section is completed may be provided. When the third set proceeds, the exercise intensity applied in the third set may be determined based on a result of comparing the heart rate of the user measured in the second set and the reference heart rate. At each of exercise time points 1435 in the sprint section and each of exercise time points 1436 in the recovery section in the third set, a guide voice for guiding the walking speed and/or strides of the user may be provided. At each of a time point 1450 at which the sprint section is completed and a time point 1452 at which the third set is completed, the exercise mode may be changed. In the middle of the third set, a guide voice for encouraging the user to exercise and/or a guide voice for informing the user of exercise information performed during the set may be provided.

A fourth set may start after the third set is completed. When the fourth set proceeds, the exercise intensity applied in the fourth set may be determined based on a result of comparing the heart rate of the user measured in the third set and the reference heart rate. At each of exercise time points 1437 in the sprint section in the fourth set, a guide voice for guiding the walking speed and/or strides of the user may be provided. At a time point 1454 at which the sprint section is completed, the exercise mode may be changed. In the middle of the fourth set, a guide voice for encouraging the user to exercise may be provided. When all sets including the fourth set are completed, a guide voice for informing the user of the completion of the exercise may be provided.

According to an embodiment, the total exercise time may extend depending on the heart rate measured during the exercise of the user. For example, when an average value of heart rates of the user measured from the first set to the sprint section of the fourth set does not reach the desired reference heart rate (or the heart rate range or heart rate zone), a fifth set may be additionally performed as shown in FIG. 14C. Referring to FIG. 14C, when the average value of heart rates of the user measured up to the time point 1454 at which the sprint section of the fourth set is completed does not reach the reference heart rate (or the heart rate range or heart rate zone), a guide voice for guiding extension of the exercise time may be provided.

After that, at each of exercise time points 1438 in a recovery section of the fourth set, a guide voice for guiding the walking speed and/or strides of the user may be provided. At each of the time point 1454 at which the sprint section is completed and a time point 1455 at which the fourth set is completed, the exercise mode may be changed.

The fifth set may start after the fourth set is completed. When the fifth set proceeds, the exercise intensity applied in the fifth set may be determined based on a result of comparing the heart rate of the user measured in the fourth set and the reference heart rate. At each of exercise time points 1439 in a sprint section of the fifth set, a guide voice for guiding the walking speed and/or strides of the user may be provided. At a time point 1456 at which the sprint section is completed, the exercise mode may be changed. In the middle of the fifth set, a guide voice for encouraging the user to exercise may be provided. When all sets including the fifth set are completed, a guide voice for informing the user of the completion of the exercise may be provided.

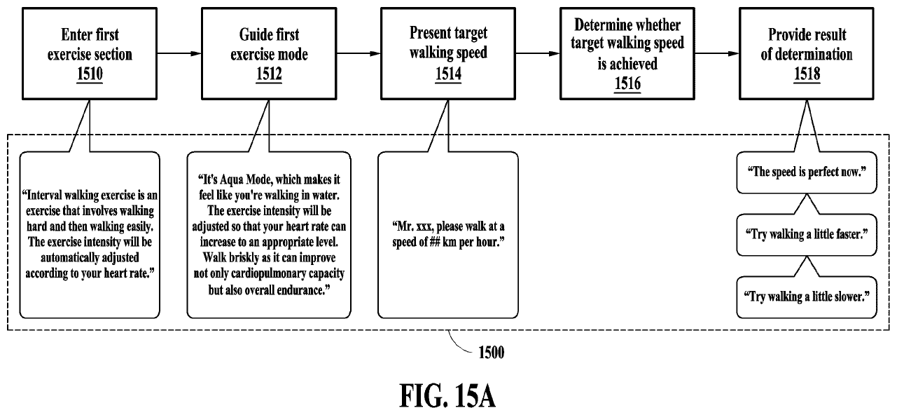

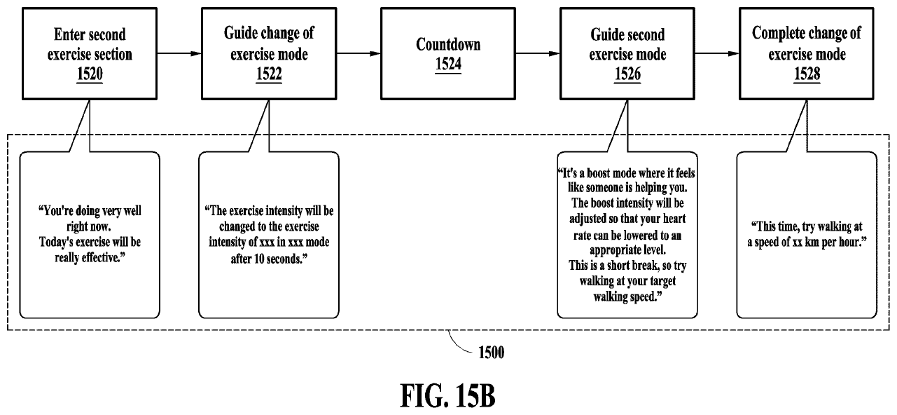

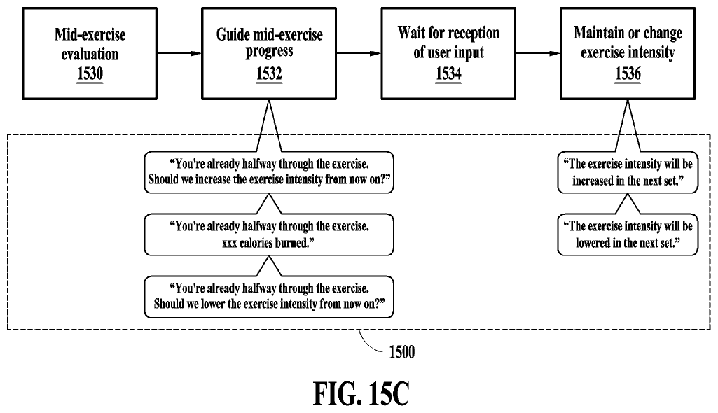

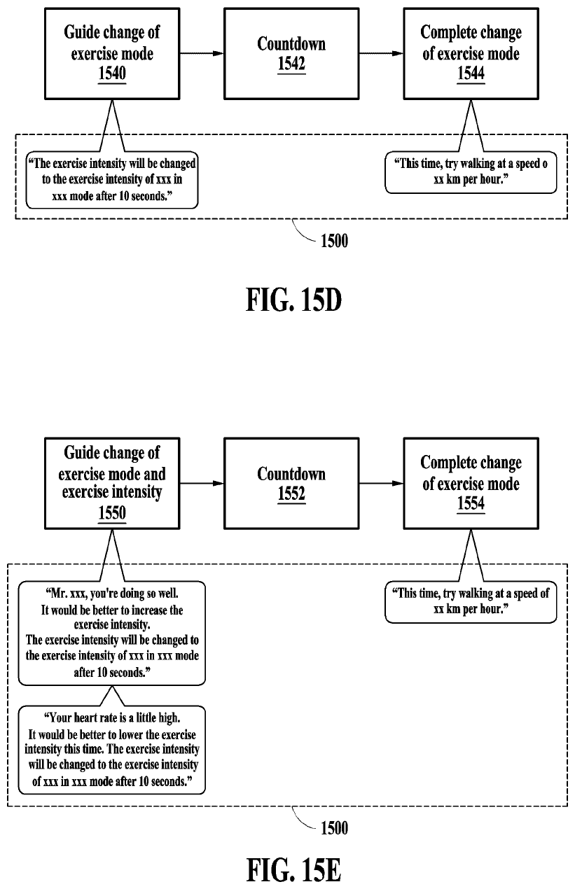

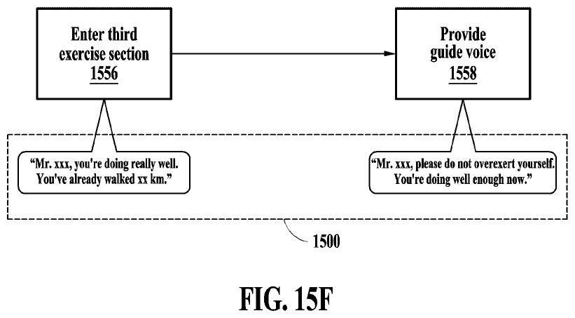

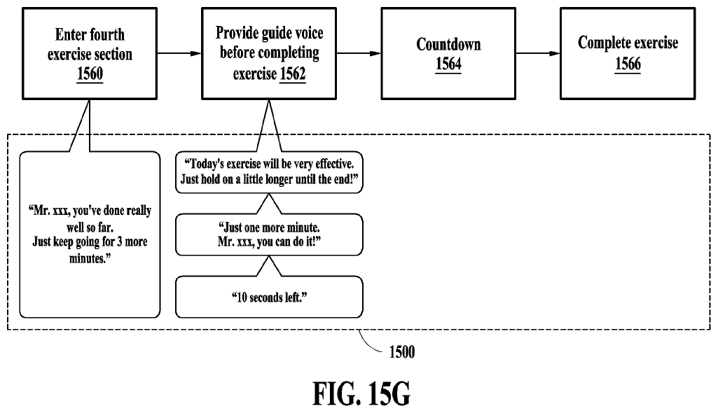

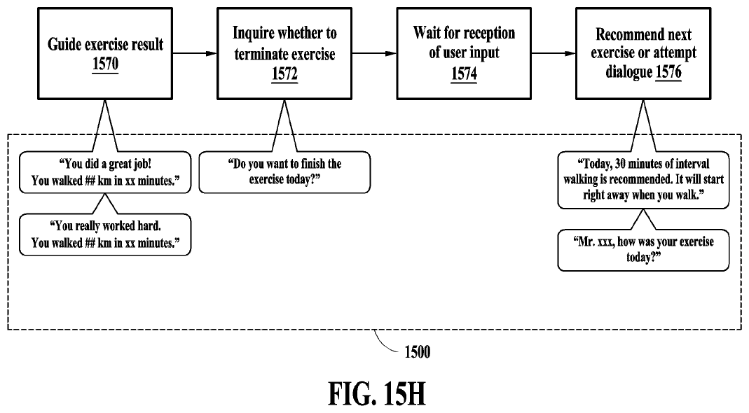

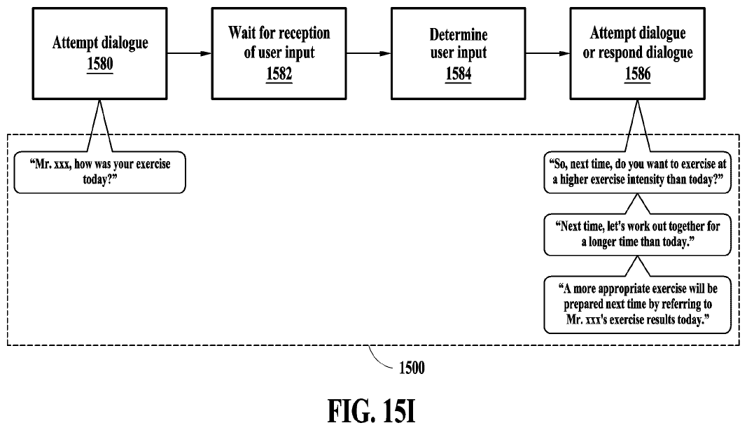

FIGS. 15A, 15B, 15C, 15D, 15E, 15F, 15G, 15H, and 15I are diagrams illustrating a voice coaching function provided for each exercise operation and situation of a user according to an embodiment.

FIG. 15A illustrates examples of operations performed in a first exercise section (e.g., the first set) after the exercise of the user starts, and guide voices 1500 provided to the user in each operation according to an embodiment. In the first exercise section, operation 1510 of entering the first exercise section, operation 1512 of guiding the first exercise mode, operation 1514 of presenting a target walking speed, operation 1516 of determining whether the target walking speed is achieved, and operation 1518 of providing a result of determining whether the target walking speed is achieved may be performed. According to an embodiment, operation 1512 may be omitted. For example, when a corresponding exercise has been performed several times before, operation 1512 may be omitted. Based on the result of determining whether the walking speed of the user is faster or slower than the target walking speed in operation 1516, a guide voice for guiding the walking speed of the user may be provided in operation 1518.

FIG. 15B illustrates examples of operations performed in a second exercise section (e.g., the second set) after the first exercise section, and guide voices 1500 provided to the user in each operation according to an embodiment. In the second exercise section, operation 1520 of entering the second exercise section, operation 1522 of changing the exercise mode, operation 1524 of countdown, operation 1526 of guiding the second exercise mode, and operation 1528 of completing the change of the exercise mode may be performed. According to an embodiment, operation 1526 may be omitted. For example, when a corresponding exercise has been performed several times before, operation 1526 may be omitted.

FIG. 15C illustrates examples of operations performed after the second exercise section and guide voices 1500 provided to the user in each operation according to an embodiment. Here, it is assumed that the time point at which the second exercise section is completed corresponds to the time point at which half of the entire exercise section is completed. After the second exercise section is completed, operation 1530 of evaluating in the middle of the exercise, operation 1532 of guiding mid-exercise progress, operation 1534 of waiting for reception of a user input, and operation 1536 of maintaining or changing the exercise intensity based on the user input may be performed. When operation 1530 of evaluating in the middle of the exercise is completed, the exercise intensity to be applied in a third exercise section may be determined based on the result of evaluating in the middle of the exercise, and a guide voice for checking whether to change the exercise intensity may be provided. The user may input a user input to allow or deny the change of the exercise intensity, for example, through a voice input, and it may be determined whether to maintain the exercise intensity or change the exercise intensity according to the user input.

FIG. 15D illustrates examples of operations, when the exercise mode is changed while the exercise intensity is not changed, and guide voices 1500 provided to the user in each operation according to an embodiment. In the above case, operation 1540 of guiding the change of the exercise mode, operation 1542 of countdown, and operation 1544 of completing the change of the exercise mode may be performed.

FIG. 15E illustrates examples of operations, when both the exercise mode and the exercise intensity are changed, and guide voices 1500 provided to the user in each operation according to an embodiment. In the above case, operation 1550 of changing the exercise mode and the exercise intensity, operation 1552 of countdown, and operation 1554 of completing the change of the exercise mode may be performed.

FIG. 15F illustrates examples of operations performed in the third exercise section (e.g., the third set) after the second exercise section, and guide voices 1500 provided to the user in each operation according to an embodiment. In the third exercise section, operation 1556 of entering the third exercise section, and operation 1558 of providing a guide voice for encouraging the exercise of the user may be performed. The guide voice may be provided when the third exercise section is completed. When the third exercise section starts, it may be determined whether to change the exercise intensity, and the operations described with reference to FIG. 15D or 15E may be performed according to the determination.

In the second exercise section and the third exercise section, a guide voice for coaching the heart rate, walking speed, and/or strides or a guide voice for encouraging the exercise of the user may be provided.

FIG. 15G illustrates examples of operations performed in a fourth exercise section (e.g., the fourth set) after the third exercise section, and guide voices 1500 provided to the user in each operation according to an embodiment. In the fourth exercise section, operation 1560 of entering the fourth exercise section, operation 1562 of providing a guide voice before completing the exercise, operation 1564 of countdown, and operation 1566 of completing the exercise may be performed. In operation 1562 of providing a guide voice before completing the exercise, for example, guide voices for informing remaining time, such as 2 minutes before, 1 minute before, or 10 seconds before completing the exercise may be provided. Also, a guide voice for encouraging the exercise may be provided.

FIG. 15H illustrates examples of operations performed after all of the exercise sections including the fourth exercise section are completed, and guide voices 1500 provided to the user in each operation according to an embodiment. After all of the exercise sections are completed, operation 1570 of guiding an exercise result, operation 1572 of inquiring whether to terminate the exercise, operation 1574 of waiting for reception of a user input, and operation 1576 of recommending a next exercise or attempting a dialogue may be performed. In operation 1570 of guiding the exercise result, a guide voice for informing a result of the exercise of the user performed in all of the exercise sections may be provided. In operation 1572 of inquiring whether to terminate the exercise, it may be inquired whether to terminate the exercise to the user, and in operation 1574 of waiting for reception of a user input, the reception of a user input (e.g., a voice input) for the corresponding inquiry may be waited. When the user input indicating that the user wants to terminate the exercise is received, a guide voice for attempting the dialogue with the user may be output. When a user input indicating that the user wants to continue exercising without terminating the exercise is received, a guide voice for recommending a next exercise may be output.

FIG. 15I illustrates examples of subsequent operations performed when the user input indicating that the user wants to terminate the exercise is received after the completing the exercise, and guide voices 1500 provided to the user in each operation according to an embodiment. In the above case, operation 1580 of attempting a dialogue, operation 1582 of waiting for reception of a user input, operation 1584 of determining the user input, and operation 1586 of attempting a dialogue or responding the dialogue may be performed. In operation 1580 of attempting the dialogue, a guide voice for attempting the dialogue with the user may be output. In operation 1582 of waiting for reception of the user input, the reception of a user input (e.g., a voice input) in response to the guide voice may be waited. When the user input is received, in operation 1584 of determining the user input, the user input may be analyzed, and in operation 1586 of attempting the dialogue or responding the dialogue, a guide voice for attempting the dialogue or a guide voice for responding the dialogue with the user may be output based on a result of the analysis.

It should be appreciated that various embodiments of the present disclosure and the terms used therein are not intended to limit the technological features set forth herein to particular embodiments and include various changes, equivalents, or replacements for a corresponding embodiment. In connection with the description of the drawings, like reference numerals may be used for similar or related components. It is to be understood that a singular form of a noun corresponding to an item may include one or more of the things, unless the relevant context clearly indicates otherwise. As used herein, “A or B”, “at least one of A and B”, “at least one of A or B”, “A, B or C”, “at least one of A, B and C”, and “at least one of A, B, or C,” each of which may include any one of the items listed together in the corresponding one of the phrases, or all possible combinations thereof. Terms, such as “first” or “second”, are simply used to distinguish a component from another component and do not limit the components in other aspects (e.g., importance or sequence). It is to be understood that if an element (e.g., a first element) is referred to, with or without the term “operatively” or “communicatively,” as “coupled with,” “coupled to,” “connected with,” or “connected to” another element (e.g., a second element), it means that the element may be coupled with the other element directly (e.g., by wire), wirelessly, or via at least a third element(s). Thus, “connected” as used herein covers both direct and indirect connections.

As used in connection with various embodiments of the disclosure, the term “module” may include a unit implemented in hardware, software, or firmware, and may interchangeably be used with other terms, for example, “logic,” “logic block,” “part,” or “circuitry”. A module may be a single integral component, or a minimum unit or part thereof, adapted to perform one or more functions. For example, according to an embodiment, the module may be implemented in a form of an application-specific integrated circuit (ASIC). Thus, each “module” herein may comprise circuitry.

The software may include a computer program, a piece of code, an instruction, or some combination thereof, to independently or uniformly instruct or configure the processing device to operate as desired. Software and data may be embodied permanently or temporarily in any type of machine, component, physical or virtual equipment, or computer storage medium or device capable of providing instructions or data to or being interpreted by the processing device. The software also may be distributed over network-coupled computer systems so that the software is stored and executed in a distributed fashion. The software and data may be stored by one or more non-transitory computer-readable recording mediums. Embodiments as set forth herein may be implemented as software including one or more instructions that are stored in a storage medium (e.g., the memory 514) that is readable by a machine. For example, a processor of the machine may invoke at least one of the one or more instructions stored in the storage medium and execute it. This allows the machine to be operated to perform at least one function according to the at least one instruction invoked. The one or more instructions may include code generated by a compiler or code executable by an interpreter. The machine-readable storage medium may be provided in the form of a non-transitory storage medium. Here, the term “non-transitory” simply means that the storage medium is a tangible device, and does not include a signal (e.g., an electromagnetic wave), but this term does not differentiate between where data is semi-permanently stored in the storage medium and where the data is temporarily stored in the storage medium.

According to an example embodiment, a method according to embodiments of the disclosure may be included and provided in a computer program product. The computer program product may be traded as a product between a seller and a buyer. The computer program product may be distributed in the form of a machine-readable storage medium (e.g., compact disc read-only memory (CD-ROM)), or be distributed (e.g., downloaded or uploaded) online via an application store (e.g., PlayStore™), or between two user devices (e.g., smartphones) directly. If distributed online, at least part of the computer program product may be temporarily generated or at least temporarily stored in the machine-readable storage medium, such as memory of the manufacturer's server, a server of the application store, or a relay server.

According to embodiments, each component (e.g., a module or a program) of the above-described components may include a single entity or multiple entities, and some of the multiple entities may be separately disposed in different components. According to embodiments, one or more of the above-described components may be omitted, or one or more other components may be added. Alternatively or additionally, a plurality of components (e.g., modules or programs) may be integrated into a single component. In such a case, the integrated component may still perform one or more functions of each of the plurality of components in the same or similar manner as they are performed by a corresponding one of the plurality of components before the integration. According to various embodiments, operations performed by the module, the program, or another component may be carried out sequentially, in parallel, repeatedly, or heuristically, or one or more of the operations may be executed in a different order or omitted, or one or more other operations may be added.

While the disclosure has been illustrated and described with reference to various embodiments, it will be understood that the various embodiments are intended to be illustrative, not limiting. It will further be understood by those skilled in the art that various changes in form and detail may be made without departing from the true spirit and full scope of the disclosure, including the appended claims and their equivalents. It will also be understood that any of the embodiment(s) described herein may be used in conjunction with any other embodiment(s) described herein.

1. A electronic device comprising:
a communication module, comprising communication circuitry, configured to communicate with a wearable device; and
a processor, comprising processing circuitry, configured to control an exercise of a user performed using the wearable device based on the communication with the wearable device, and to:
output a first guide voice for guiding proper wearing of the wearable device when connection to the wearable device is detected;
output a second guide voice for guiding an operation for sensor initialization of the wearable device after first notification data indicating that the user is properly wearing the wearable device is received from the wearable device;
output a third guide voice for inquiring whether to select a current recommended exercise program, among a plurality of recommended exercise programs, after second notification data indicating that the sensor initialization is completed is received from the wearable device; and
execute an exercise mode according to a target recommended exercise program in response to reception of response data corresponding to selection of the target recommended exercise program among the recommended exercise programs from the wearable device.

2. The electronic device of claim 1, wherein the processor is configured to:
determine whether the user has performed a gesture motion corresponding to the selection of the target recommended exercise program based on motion data of the user received from the wearable device; and
determine a recommended exercise program that is guided through the third guide voice as the target recommended exercise program when it is determined that the gesture motion has been performed.

3. The electronic device of claim 1, wherein the processor is configured to:
output the third guide voice for inquiring selection of a first recommended exercise program among the recommended exercise programs;
output the third guide voice for inquiring selection of a second recommended exercise program among the recommended exercise programs when response data corresponding to the selection of the first recommended exercise program is not received during a predefined period of time; and
output the third guide voice for inquiring selection of a third recommended exercise program among the recommended exercise programs when response data corresponding to the selection of the second recommended exercise program is not received during a predefined period of time.

4. The electronic device of claim 1, wherein the processor is configured to:
output the third guide voice for inquiring whether to select a next recommended exercise program in at least one of a case where motion data of the user is not received from the wearable device and a case where a gesture motion corresponding to refusal of the current recommended exercise program is recognized during a predefined period of time after the third guide voice for inquiring selection of the current recommended exercise program is output.

5. The electronic device of claim 1, wherein the processor is configured to:
determine whether an exercise state is a stop of an exercise based on motion data of the user received through the communication module; and
output a fourth guide voice for inquiring whether to terminate the exercise to the user when the exercise state is determined as the stop of the exercise.

6. The electronic device of claim 5, wherein the processor is configured to:
provide the user with exercise result data about the exercise performed by the user after response data corresponding to selection of the stop of the exercise is received from the wearable device.

7. The electronic device of claim 6, wherein the processor is configured to:
determine whether the user has performed a gesture motion corresponding to the selection of the stop of the exercise based on motion data of the user received from the wearable device; and
determine that the exercise of the user is completed when it is determined that the gesture motion has been performed.

8. The electronic device of claim 1, further comprising:
a display module,
wherein the processor is configured to:
output a list of other recommended exercise programs through the display module when the response data corresponding to the selection of the target recommended exercise program is not received until an output of a third guide voice for sequentially inquiring whether to select each of the recommended exercise programs is completed.

9. The electronic device of claim 1, wherein the processor is configured to:
provide a guide voice for exercise coaching to the user based on an exercise situation and a physical condition of the user during the exercise of the user.

10. An interaction method between an electronic device and a wearable device, performed by the electronic device, the interaction method comprising:
outputting a first guide voice for guiding proper wearing of the wearable device when connection to the wearable device is detected;
receiving first notification data indicating that a user is properly wearing the wearable device from the wearable device;
outputting a second guide voice for guiding an operation for sensor initialization of the wearable device after the first notification data is received;
receiving second notification data indicating that the sensor initialization is completed from the wearable device;
outputting a third guide voice for inquiring whether to select a current recommended exercise program among a plurality of recommended exercise programs after the second notification data is received; and
executing an exercise mode according to a target recommended exercise program when response data corresponding to selection of the target recommended exercise program among the recommended exercise programs is received from the wearable device.

11. An electronic device comprising:
a communication module, comprising communication circuitry, configured to communicate with a wearable device; and
a processor, comprising processing circuitry, configured to control to provide a guide voice for exercise coaching and/or guidance to a user based on a physical condition of the user during an exercise of the user performed using the wearable device.

12. The electronic device of claim 11, wherein the processor is configured to:
control to provide a guide voice indicating to lower an exercise intensity of the exercise in response to a heart rate of the user being continuously maintained to be higher than a reference heart rate; and
control to provide a guide voice indicating to increase the exercise intensity of the exercise in response to the heart rate of the user being continuously maintained to be lower than a reference heart rate section.

13. The electronic device of claim 11, wherein the processor is configured to:
determine to what extent the heart rate of the user satisfies the reference heart rate section, and control to provide a guide voice for changing the exercise intensity according to a result of the determination.

14. The electronic device of claim 11, wherein the processor is configured to:
control to provide the user with a guide voice according to a walking speed of the user or a stride of the user; and
control to provide the user with a guide voice for changing the exercise intensity when an exercise section of the exercise is changed.

15. An electronic device comprising:
a communication module, comprising communication circuitry, configured to communicate with a wearable device; and
a processor, comprising processing circuitry, configured to control an exercise of a user performed using the wearable device based on the communication with the wearable device, and to:
output a first guide voice for guiding proper wearing of the wearable device when connection to the wearable device is detected;
output a second guide voice for guiding an operation for sensor initialization of the wearable device after first notification data indicating that the user is properly wearing the wearable device is received from the wearable device;
output a list of exercise programs through a screen after second notification data indicating that the sensor initialization is completed is received from the wearable device; and
control to execute an exercise mode according to an exercise program selected through a user input from the output list of the exercise programs.
